(19) United States
(12) Patent Application Publication
(10) Pub. No.: US 2020/0268265 A1
(43) Pub. Date: Aug. 27, 2020

(54) INTEGRATION OF SENSOR-BASED CARDIOVASCULAR MEASURES INTO PHYSICAL BENEFIT MEASURE RELATED TO HEARING INSTRUMENT USE

(71) Applicant: Starkey Laboratories, Inc., Eden Prairie, MN (US)

(72) Inventors: Kyle Walsh, Minneapolis, MN (US); Thomas Scheller, Eden Prairie, MN (US); Christopher L. Howes, Eden Prairie, MN (US); David A. Fabry, Eden Prairie, MN (US)

(21) Appl. No.: 16/800,948

(22) Filed: Feb. 25, 2020

Related U.S. Application Data
(60) Provisional application No. 62/810,298, filed on Feb. 25, 2019.
(60) Provisional application No. 62/854,710, filed on May 30, 2019.

(51) Int. Cl. A61B 5/024; A61B 5/12; A61B 5/00; H04R 25/00

(52) U.S. Cl. CPC A61B 5/02416; A61B 5/12; A61B 5/74; H04R 25/558; A61B 5/0006; H04R 25/70; H04R 25/554; A61B 5/0022

(57) ABSTRACT

A computing system receives data from one or more hearing instruments. Additionally, the computing system determines, based on the data received from the one or more hearing instruments, a heart health measure for a user of the one or more hearing instruments. The heart health measure is an indication of one or more aspects of a health of a heart of the user. The computing system may output an indication of the heart health measure.

This patent application claims the benefit of U.S. Provisional Patent Application No. 62/810,298, filed Feb. 25, 2019, and U.S. Provisional Patent Application No. 62/854,710, filed May 30, 2019, the entire content of each of which is incorporated by reference.

### TECHNICAL FIELD

This disclosure relates to hearing instruments.

### BACKGROUND

In many people, hearing loss is a gradual process that occurs over many years. As a result, many people grow accustomed to living with reduced hearing without recognizing the auditory experiences and opportunities they are missing. For example, a person might not realize how much less conversation he or she engages in due to his or her hearing loss. As a result of hearing loss, reduced audibility, and reduced social interaction, patients also experience follow-on effects such as dementia, depression, and generally poorer health.

### SUMMARY

This disclosure describes a computing system that receives data from one or more hearing instruments. Additionally, the computing system determines, based on the data received from the one or more hearing instruments, a heart health measure for a user of the one or more hearing instruments. The heart health measure is an indication of one or more aspects of a health of a heart of the user. The computing system may output an indication of the heart health measure. Other examples of this disclosure use data received from the one or more hearing instruments to determine levels of emotional stress. Still other examples of this disclosure use data received from the one or more hearing instruments to determine whether the user has fallen.

In one example, this disclosure describes a computer-implemented method comprising: receiving, by a computing system comprising a set of one or more electronic computing devices, heart-related data from one or more hearing instruments; determining, by the computing system, based on the heart-related data received from the one or more hearing instruments, a heart health measure for a user of the one or more hearing instruments, the heart health measure being an indication of one or more aspects of a health of a heart of the user; and outputting, by the computing system, an indication of the heart health measure to the user of the hearing instruments.

In another example, this disclosure describes a computer-implemented method comprising: receiving, by a computing system comprising one or more electronic computing devices, stress-related data from one or more hearing instruments; determining, by the computing system, based on the stress-related data, an emotional stress measure of a user of the one or more hearing instruments, the emotional stress measure being an indication of one or more aspects of a level of emotional stress of the user; and outputting, by the computing system, an indication of the emotional stress measure to the user of the hearing instruments.

In another example, this disclosure describes a computer-implemented method comprising: obtaining, by a computing system, physiological data based on signals generated by a first set of one or more sensors of a hearing instrument, wherein the physiological data includes heart-related data; modifying, by the computing system, a sensitivity level of a fall detection algorithm based on the physiological data; and performing, by the computing system, the fall detection algorithm to determine, based on signals from a second set of one or more sensors of the hearing instrument, whether a user of the hearing instrument has fallen.

In another example, this disclosure describes a computer-implemented method comprising: determining, by a computing system, whether a user of a hearing instrument has fallen; based on a determination that the user has fallen, activating, by the computing system, one or more sensors of the hearing instrument that generate heart-related data regarding the user; determining, by the computing system, based on the heart-related data, whether to prompt the user to confirm that the user has fallen; and based on a determination to prompt the user to confirm that the user has fallen, causing, by the computing system, the hearing instrument to generate a message prompting the user to confirm that the user has fallen.

This disclosure also describes examples of computing systems having one or more processors configured to perform the methods. Also described are computer-readable storage media having instructions for causing computer systems to perform the methods.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a conceptual diagram illustrating an example system that includes hearing instruments, in accordance with one or more techniques of this disclosure.

FIG. 2 is a conceptual diagram illustrating contributions of sub-components to a cognitive benefit measure, in accordance with one or more aspects of this disclosure.

FIG. 3 is a block diagram illustrating example components of a hearing instrument, in accordance with one or more aspects of this disclosure.

FIG. 4 is a block diagram illustrating example components of a mobile computing device, in accordance with one or more aspects of this disclosure.

FIG. 5 is a flowchart illustrating an example operation of a computing system, in accordance with one or more aspects of this disclosure.

FIG. 6 is a flowchart illustrating an example operation to compute a body fitness measure in accordance with one or more aspects of this disclosure.

FIG. 7 is a flowchart illustrating an example operation to compute a wellness measure in accordance with one or more aspects of this disclosure.

FIG. 8 is an example graphical user interface (GUI) for display of a cognitive benefit measure in accordance with one or more aspects of this disclosure.

FIG. 9 is an example GUI for display of a body fitness measure in accordance with one or more aspects of this disclosure.

FIG. 10 is an example GUI for display of a wellness measure in accordance with one or more aspects of this disclosure.

FIG. 11 is a block diagram illustrating example components of a hearing instrument, in accordance with one or more aspects of the disclosure.

FIG. 12 is an example GUI for display of a wellness measure in accordance with one or more aspects of this disclosure.

FIG. 13 is an example GUI for display of a heart health interface in accordance with one or more aspects of this disclosure.

FIG. 14A is a conceptual diagram illustrating a variant of the GUI of FIG. 12, in accordance with one or more aspects of this disclosure.

FIG. 14B is a conceptual diagram illustrating a variant of the GUI of FIG. 12, in accordance with one or more aspects of this disclosure.

FIG. 15 is a conceptual diagram illustrating an example calculation technique for a body score, in accordance with one or more aspects of this disclosure.

FIG. 16 is a conceptual diagram illustrating an example calculation technique for a body score, in accordance with one or more aspects of this disclosure.

FIG. 17 is a conceptual diagram illustrating an example user interface feature for indicating values of components of heart health sub-component, in accordance with one or more aspects of this disclosure.

FIG. 18 is a conceptual diagram illustrating an example user interface feature for indicating values of components of heart health sub-component, in accordance with one or more aspects of this disclosure.

FIG. 19 is a conceptual diagram illustrating an example user interface feature for a goal-based heart health component in accordance with one or more aspects of this disclosure.

FIG. 20 is a conceptual diagram illustrating an example expanded activity sub-component in which heart rate measurements are indicated for different types of activities in accordance with one or more aspects of this disclosure.

FIG. 21 is a flowchart illustrating an example operation, in accordance with one or more aspects of this disclosure.

FIG. 22 is a flowchart illustrating an example operation, in accordance with one or more aspects of this disclosure.

FIG. 23 is a flowchart of an example operation in which a computing system uses stress-related data, in accordance with one or more aspects of this disclosure.

FIG. 24 is a flowchart illustrating an example operation in which a computing system uses stress-related data to determine whether to perform an intervention action, in accordance with one or more aspects of this disclosure.

FIG. 25 is a flowchart illustrating an example operation, in accordance with one or more aspects of this disclosure.

FIG. 26 is a flowchart illustrating an example operation, in accordance with one or more aspects of this disclosure.

### DETAILED DESCRIPTION

In this disclosure, ordinal terms such as “first,” “second,” “third,” and so on, are not necessarily indicators of positions within an order, but rather may be used to distinguish different instances of the same thing. Examples provided in this disclosure may be used together, separately, or in various combinations.

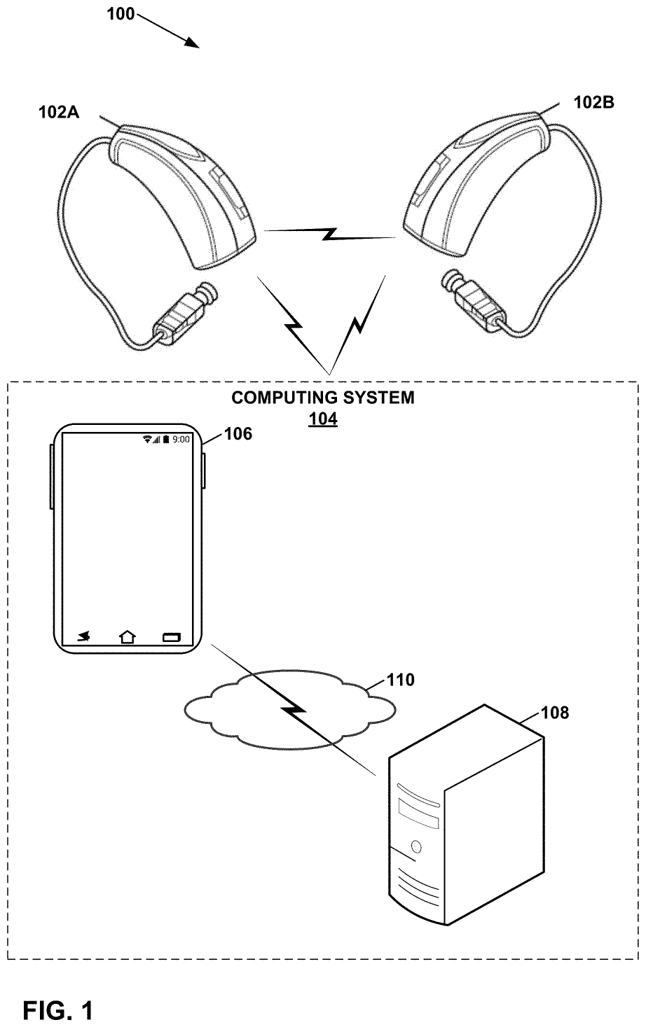

FIG. 1 is a conceptual diagram illustrating an example system 100 that includes hearing instruments 102A, 102B, in accordance with one or more techniques of this disclosure. This disclosure may refer to hearing instruments 102A and 102B collectively, as “hearing instruments 102.” A user may wear hearing instruments 102. In some instances, such as when the user has unilateral hearing loss, the user may wear a single hearing instrument. In other instances, such as when the user has bilateral hearing loss, the user may wear two hearing instruments, with one hearing instrument for each ear of the user. In the example of FIG. 1, system 100 comprises hearing instruments 102 and a computing system 104.

Additionally, system 100 includes a computing system 104. Computing system 104 comprises one or more electronic devices. For instance, in the example of FIG. 1, computing system 104 comprises a mobile device 106, a server device 108, and a communication network 110. In other examples, mobile device 106 may be replaced with one or more other types of devices, such as accessory devices, access gateways, and so on.

Hearing instruments 102 may comprise one or more of various types of devices that are configured to provide auditory stimuli to a user and that are designed for wear and/or implantation at, on, or near an ear of the user. Hearing instruments 102 may be worn, at least partially, in the ear canal or concha. One or more of hearing instruments 102 may include behind the ear (BTE) components that are worn behind the ears of the user. In some examples, hearing instruments 102 comprise devices that are at least partially implanted into or osseointegrated with the skull of the user. In some examples, one or more of hearing instruments 102 is able to provide auditory stimuli to the user via a bone conduction pathway.

In any of the examples of this disclosure, each of hearing instruments 102 may comprise a hearing assistance device. Hearing assistance devices include devices that help a user hear sounds in the user's environment. Example types of hearing assistance devices may include hearing aid devices, Personal Sound Amplification Products (PSAPs), cochlear implant systems (which may include cochlear implant magnets, cochlear implant transducers, and cochlear implant processors), and so on. In some examples, hearing instruments 102 are over-the-counter, direct-to-consumer, or prescription devices. Furthermore, in some examples, hearing instruments 102 include devices that provide auditory stimuli to the user that correspond to artificial sounds or sounds that are not naturally in the user's environment, such as recorded music, computer-generated sounds, or other types of sounds. For instance, hearing instruments 102 may include so-called “hearables,” earbuds, earphones, or other types of devices. Some types of hearing instruments provide auditory stimuli to the user corresponding to sounds from the user's environmental and also artificial sounds.

In some examples, one or more of hearing instruments 102 includes a housing or shell that is designed to be worn in the ear for both aesthetic and functional reasons and encloses the electronic components of the hearing instrument. Such hearing instruments may be referred to as in-the-ear (ITE), in-the-canal (ITC), completely-in-the-canal (CIC), or invisible-in-the-canal (IIC) devices. In some examples, one or more of hearing instruments 102 may be behind-the-ear (BTE) devices, which include a housing worn behind the ear contains all of the electronic components of the hearing instrument, including the receiver (i.e., the speaker). The receiver conducts sound to an earbud inside the ear via an audio tube. In some examples, one or more of hearing instruments 102 may be receiver-in-canal (RIC) hearing-assistance devices, which include a housing worn behind the ear that contains electronic components and a housing worn in the ear canal that contains the receiver. The techniques of this disclosure are not limited to the form of the hearing instrument, mobile device, or server device shown in FIG. 1.

Hearing instruments 102 may be configured to communicate with each other. For instance, in any of the examples of this disclosure, hearing instruments 102 may communicate with each other using one or more wirelessly communication technologies. Example types of wireless communication technology include Near-Field Magnetic Induction (NFMI) technology, a 900 MHz technology, a BLUETOOTH™ technology, a WI-FI™ technology, audible sound signals, ultrasonic communication technology, infrared communication technology, an inductive communication technology, or another type of communication that does not rely on wires to transmit signals between devices. In some examples, hearing instruments 102 use a 2.4 GHz frequency band for wireless communication. In examples of this disclosure, hearing instruments 102 may communicate with each other via non-wireless communication links, such as via one or more cables, direct electrical contacts, and so on.

Hearing instruments 102 are configured to communicate wirelessly with computing system 104. For example, hearing instruments 102 and computing system 104 may communicate wirelessly using a BLUETOOTH™ technology, a WIFI™ technology, or another type of wireless communication technology. In the example of FIG. 1, hearing instruments 102 may communicate wirelessly with mobile device 106. In some examples, hearing instruments 102 may use a 2.4 GHz frequency band for wireless communication with mobile device 106 or other computing devices.

Mobile device 106 may communicate with server device 108 via communication network 110. Communication network 110 may comprise one or more of various types of communication networks, such as cellular data networks, WIFI™ networks, the Internet, and so on. Mobile device 106 may communicate with server device 108 to store data to and retrieve data from server device 108. Thus, from the perspective of mobile device 106 and hearing instruments 102, server device 108 may be considered to be in the “cloud.”

Hearing instruments 102 may implement a variety of features that help a wearer of hearing instruments 102 hear better. For example, hearing instruments 102 may amplify the intensity of incoming sound, amplify the intensity of certain frequencies of the incoming sound, or translate or compress frequencies of the incoming sound. In another example, hearing instruments 102 may implement a directional processing mode in which hearing instruments 102 selectively amplifies sound originating from a particular direction (e.g., to the front of the wearer) while potentially fully or partially canceling sound originating from other directions. In other words, a directional processing mode may selectively attenuate off-axis unwanted sounds. The directional processing mode may help wearers understand conversations occurring in crowds or other noisy environments. In some examples, hearing instruments 102 may reduce noise by canceling out or attenuating certain frequencies. Furthermore, in some examples, hearing instruments 102 may help a wearer enjoy audio media, such as music or sound components of visual media, by outputting sound based on audio data wirelessly transmitted to hearing instruments 102.

As previously mentioned, a person may lose their hearing gradually over the course of many years. Because hearing loss may be a slow process, a person who is gradually losing his or her hearing may grow accustomed to living with impaired hearing and not realize the value added to the person's life by being able to fully access the auditory environment. For instance, the person may not realize how much less time he or she spends in conversation or enjoying audio media because of the person's hearing loss. This may remain true even after a person acquires a hearing instrument. That is, because a person having a hearing instrument does not always wear the hearing instrument, the person may not realize the extent to which the hearing instrument enhances his or her life while wearing the hearing instrument as opposed to when the person is not wearing the hearing instrument.

Research has shown that people who more frequently interact with others and their environments tend to have better cognitive skills and better emotional health, both of which may lead to better health outcomes. However, depression and physical inactivity may be more common among people who seldom converse with others. This problem may be especially acute for older people, who are more likely to have hearing loss.

In accordance with one or more techniques of this disclosure, a cognitive benefit measure is calculated based on data collected by hearing instruments 102. In some examples, the cognitive benefit measure is an indication of a change of a cognitive benefit of the wearer of hearing instruments 102 attributable to use of hearing instruments 102 by the wearer of hearing instruments 102. In some examples, hearing instruments 102 calculate the cognitive benefit measure. In other examples, the cognitive benefit measure is calculated by one or more computing devices of computing system 104. For instance, in the example of FIG. 1, mobile device 106 or server device 108 may calculate the cognitive benefit measure. For ease of explanation, many of the examples of this disclosure describe computing system 104 calculating the cognitive benefit measure. However, these examples can be adapted to scenarios where hearing instruments 102 calculates the cognitive benefit measure.

As described herein, computing system 104 may calculate a cognitive benefit measure for a wearer of hearing instruments 102 based on a plurality of sub-components of the cognitive benefit measure. For example, as part of determining the cognitive benefit measure, computing system 104 may determine a plurality of sub-components of the cognitive benefit measure and may determine the cognitive benefit measure based on the plurality of sub-components of the cognitive benefit measure. In some examples, hearing instruments 102 determines one or more of the sub-components of the cognitive benefit measure. In some examples, the sub-components include one or more of an “audibility” sub-component, an “intelligibility” sub-component, a “comfort” sub-component, a “focus” sub-component, a “sociability” sub-component, and a “connectivity” sub-component. In some examples, each of the sub-components shares a common range (e.g., from 0 to 100), which may make combination of data efficient. In some examples, computing system 104 may reset each of the sub-components for each scoring period. For instance, computing system 104 may reset the values of the sub-components once per day or other recurrence period.

The audibility sub-component for a wearer of hearing instruments 102 is a measure of the improvement in audibility provided to the wearer by hearing instruments 102. The audibility sub-component may be considered the amount of environmental sounds that are quieter than the wearer's unaided audiometric thresholds, but that are made audible through amplification by hearing instruments 102, scaled to a range used by the other sub-components. In other words, the audibility sub-component is related to hearing more quiet sounds in the wearer's environment. To determine the audibility sub-component, computing system 104 may compare a patient's hearing thresholds to a standardized stimulus response across frequency. For instance, in some examples, the audibility sub-component is calculated by subtracting the percentage of a standardized sound stimulus (e.g., a moderate-level (65 dB SPL) long-term averaged speech input) that is audible without a hearing instrument from the percentage of sound that is audible with a hearing instrument; both percentages are calculated by dividing the number of audible frequency channels in hearing instruments 102 by the number of total channels in the device. A channel in a hearing instrument is a subset of frequencies over which the processing of incoming sound can be different from that at other frequencies. For example, a hearing aid channel may have a highpass cutoff of 1480 Hz, and a lowpass cutoff of 1720 Hz. So, the “total channels” in a hearing aid are the number of distinct divisions of frequency. An “audible channel” is one wherein the level of the input stimulus (in dB SPL) plus the gain applied to the stimulus (in dB) results in an overall level that is above the hearing threshold of the listener in that frequency range. Each of the unaided thresholds corresponds to a different frequency. A wearer of hearing instruments 102 is unable to hear the frequency corresponding to an unaided threshold if an intensity of a sound at the corresponding frequency is below the unaided threshold. In one example, audibility sub-component is calculated as a number of frequency bands made audible by hearing instruments 102 divided by a total number of frequency bands handled by hearing instruments 102. In this example, each of the frequency bands may be a contiguous range within a frequency spectrum.

The intelligibility sub-component for the wearer of hearing instruments 102 is a numerical estimate of the improvement in speech understanding provided to the wearer by hearing instruments 102. The intelligibility sub-component may be considered a measure of understanding more words in conversation. In some examples, the intelligibility sub-component is a percentage improvement in intelligibility. For instance, in one such example, the intelligibility sub-component is equal to a first value multiplied by 100, where the first value is equal to a third value subtracted from a second value. The second value is equal to an aided intelligibility score, and the third value is equal to an unaided intelligibility score. The intelligibility scores both are calculated from the Speech Intelligibility Index (SII), which is a standardized measure of intelligibility. Of course, other measures of intelligibility scaled to the same range as the other sub-components may be used.

The comfort sub-component for the wearer of hearing instruments 102 is a numerical value indicating a measure of noise reduction provided by hearing instruments 102. The comfort sub-component may be considered a measure of noise reduction in the wearer's environment. In some examples, the comfort sub-component is equal to an average or a sum of noise reduction. For instance, in one such example, the comfort sub-component is equal to a first value. In this example, the first value is equal to a sum of the average noise reduction (in dB) across memories and environments, weighted by the time spent in each memory in a set of one or more memories and each environment in a set of one or more environments, scaled to the standardized range used in the other sub-components. In this example, hearing instruments 102 comprises different memories, which have different signal processing schemes tailored to specific listening situations. For example, there is a “Restaurant” memory, a “Music” memory, and so on. Each of the environments is an acoustic situation that hearing instruments 102 classify automatically. Example types of environments include a “Speech-in-Noise” environment, a “Quiet” environment, a “Machine Noise” environment, and so on.

The focus sub-component for the wearer of hearing instruments 102 is a numerical value indicating an amount of time hearing instruments 102 have spent in a directional processing mode. The focus sub-component may be considered a measure of the wearer being able to hear sounds most important to the wearer. The focus sub-component may be scaled to be in a range used by the other sub-components. For instance, in some examples, the focus sub-component is equal to a percentage of time spent in a directional processing mode. For instance, in one such example, the focus sub-component is equal to a first value multiplied by 100, where the first value is equal to a second value divided by a third value; the second value being equal to an amount of time spent in a directional processing mode; the third value being equal to the total amount of time hearing instruments 102 is powered on. In an omni-directional mode, hearing instruments 102 do not selectively amplify or attenuate sounds from particular directions.

The sociability sub-component for the wearer of hearing instruments 102 is a numerical value indicating an amount of time hearing instruments 102 have spent in auditory environments involving speech. The sociability sub-component may be considered a measure of time spent in conversation. The sociability sub-component may be scaled to be in a range used by the other sub-components. In some examples, the sociability sub-component is a percentage of time spent in social situations. For instance, in one such example, the sociability sub-component is equal to a first value multiplied by 100, where the first value is equal to a second value divided by a third value. In this example, the second value is equal to the amount of time spent in speech and speech in noise, and the third value is equal to the total amount of time that hearing instruments 102 is powered on.

The connectivity sub-component for the wearer of hearing instruments 102 is a numerical value indicating an amount of time hearing instruments 102 have spent streaming audio data from devices that are wirelessly connective to hearing instruments 102. The connectivity sub-component may be considered a measure of time connecting with media. In some examples, the connectivity sub-component for the wearer is a measure of the amount of time spent streaming media (or the amount of time hearing instruments 102 spent maintaining connectivity for streaming media) relative to an amount of time, such as an amount of time associated with a maximum benefit attained from streaming media. This measure may be on a same scale (e.g., 0 to 100, 0 to 50, etc.) as the other sub-components. For instance, in one such example, the connectivity sub-component may be equal to a first value. In this example, the first value is equal to an amount of time spent streaming from a separate wireless device, up to a time associated with the maximum benefit attained from streaming media, divided by the time associated with the maximum benefit attained from streaming media.

Computing system 104 may determine the cognitive benefit measure based on the sub-components in various ways. For example, computing system 104 may determine the cognitive benefit measure based on an average or weighted average of the sub-components. In other words, the cognitive benefit measure may be an average of all the sub-component data, although the sub-components may be differentially weighted before averaging occurs. For example, the “connectivity” sub-component may be weighted more than the other measures because the expectation is that the connectivity sub-component typically yields a relatively small score because patients spend only a small percentage of the time streaming audio to their hearing aids. In some examples, computing system 104 determines the weights used in calculating the weighted average by normalizing the sub-components by a maximum benefit expected or predicted for each sub-component.

In some examples, computing system 104 scales the cognitive benefit measure (and the sub-components) by use time of hearing instruments 102. For example, if a user does not wear his or her hearing instrument on a given day, the cognitive benefit measure may not be calculated, but the more the user wears hearing instruments 102, the larger the cognitive benefit measure. This type of scaling may be intuitive for the user, and time spent using hearing instruments 102 may be one contributing factor to the cognitive benefit measure over which the user has the most control.

In some examples, computing system 104 may store historical cognitive benefit measures for the wearer of hearing instruments 102. For example, computing system 104 may store a cognitive benefit measure for each day or other time period. Additionally, computing system 104 may output data based on the historical cognitive benefit measures for display. In this way, the wearer of hearing instruments 102 may be able to track the wearer's cognitive benefit measures over time. For instance, the wearer of hearing instruments 102 may be able to track his or her progress.

As noted above, the cognitive benefit measure may be calculated based on data collected by hearing instruments 102. In some examples, hearing instruments 102 writes data to a data log. For example, hearing instruments 102 may store, in memory, counter data used for calculation of sub-components. For instance, hearing instruments 102 may store data indicating an amount of time hearing instruments 102 spent streaming media, an amount of time spent in a directional processing mode, and other values. Hearing instruments 102 may flush these values out to the data log on a period basis and may reset the values.

Hearing instruments 102 may communicate data in the data log to computing system 104. Computing system 104 may receive, from hearing instruments 102, the data from the data log. Computing system 104 may use the received information to determine the cognitive benefit measure.

Hearing instruments 102 may write the data to the data log on a periodic basis, e.g., once per time period. In some examples, the duration of the time period changes during the life cycle of hearing instruments 102. For example, hearing instruments 102 may write data to the data log once every 15 minutes during the first two years of use of hearing instruments 102 and once every 60 minutes following the first two years of use of hearing instruments 102. Because hearing instruments 102 send data in the data log, as opposed to the live counter data, and hearing instruments 102 may update the data log on a periodic basis, the user may be able to access an updated cognitive benefit measures at least as often as the same periodic basis.

Furthermore, in some examples, in addition to determining a cognitive benefit measure (e.g., a brain score) for the wearer of hearing instruments 102, computing system 104 may use data collected by hearing instruments 102 to determine a body fitness measure for the wearer of hearing instruments 102. The body fitness measure for the wearer of hearing instruments 102 may be an indication of physical activity in which the wearer of hearing instruments 102 engages while wearing hearing instruments 102. Like the cognitive benefit measure, computing system 104 may determine the body fitness measure based on a plurality of sub-components. For instance, computing system 104 may determine the body fitness measure based on a “steps” sub-component, an “activity” sub-component, and a “move” sub-component. The “steps” component may indicate a number of steps (e.g., while walking or running) that the wearer of hearing instruments 102 has taken during a current scoring period. The “activity” sub-component may be a measure of vigorous activity in which the wearer of hearing instruments 102 has engaged during the current scoring period. The “move” sub-component may be based on a number time of intervals during the current scoring period in which the wearer of hearing instruments 102 moves for a given amount of time. The current scoring period may be an amount of time after which computing system 104 resets the cognitive benefit measure and/or the body fitness measure. For instance, the current scoring period may be one day, one week, or another time period. Thus, the cognitive benefit measure and the body fitness measure, and sub-components thereof, may be reset periodically or recurrently.

In some examples, computing system 104 may determine values of one or more of the sub-components of the cognitive benefit measure and the body fitness measure using goals. For instance, in one example with respect to the “steps” sub-component of the body fitness measure, the wearer of hearing instrument 103 may set a number of steps to take during a scoring period as a goal for the “steps” sub-component. In this example, computing system 104 may determine the value of the “steps” component based on the progress of the wearer of hearing instruments 102 during the scoring period toward the goal for the “steps” component. In some examples, such goals may be user-configurable. For example, computing system 104 may permit a user (e.g., the wearer of hearing instruments 102, a caregiver, a health care provider, or another person) to set the goals for particular wearers of hearing instruments or for a population of patients. For example, wearers of hearing instruments may be characterized (e.g., classified) using one or more of various techniques, such as artificial intelligence using demographic or medical information. In this example, goal(s) may be determined based upon such characterizations about wearers of hearing instruments.

In some examples, computing system 104 may determine a “wellness” measure (e.g., a wellness score) for the wearer of hearing instruments 102. The wellness measure for the wearer of hearing instruments 102 may be an indication of an overall wellness of the wearer of hearing instruments 102. Computing system 104 may determine the wellness measure based on the cognitive benefit measure and the body fitness measure of the wearer of hearing instruments 102 for a scoring period. For instance, computing system 104 may determine the wellness measure as a weighted sum of the cognitive benefit measure, the body fitness measure, and possibly one or more other factors. In some examples, computing system 104 may determine the wellness measure as a multiplication product of the cognitive benefit measure and the body fitness measure.

In some examples, hearing instruments 102 calculate the body fitness measure and/or the wellness measure. In other examples, the body fitness measure and/or the wellness measure is calculated by one or more computing devices of computing system 104. For instance, in the example of FIG. 1, mobile device 106 or server device 108 may calculate the body fitness measure and/or the wellness measure. For ease of explanation, many of the examples of this disclosure describe computing system 104 calculating the body fitness measure and/or the wellness measure. However, these examples can be adapted to scenarios where hearing instruments 102 calculate the body fitness measure and/or wellness measure.

Computing system 104 may be configured to generate alerts based on one or more of a cognitive benefit measure, body fitness measure, a wellness measure of a wearer of hearing instruments 102, or a combination thereof. An alert may alert the wearer of hearing instruments 102 or another person to the occurrence or risk of occurrence of a particular condition. In other words, computing system 104 may generate, based on the cognitive benefit measure, an alert to the wearer of hearing instruments 102 or another person. Computing system 104 may transmit an alert to a caregiver, healthcare professional, family member, or other person or persons. Computing system 104 may generate an alert when one or more of various conditions occur. For example, computing system 104 may generate an alert if computing system 104 detects a consistent downward trend in the wearer's body fitness measure, cognitive benefit measure, and/or wellness measure. In another example, computing system 104 may generate an alert if computing system 104 determines that the wearer's body fitness measure, cognitive benefit measure, and/or wellness measure are below one or more thresholds for a threshold amount of time (e.g., a particular number of days). In some examples, responsive to declaration of an alert, a therapy may be changed, or additional diagnostics may be performed, encouragement may be provided, or a communication may be initiated. In other examples, hearing instruments 102 may generate the alerts.

In some examples, hearing instruments 102 does not have a real time clock that keeps track of the current time and date. Not including such a real time clock in hearing instruments 102 may be advantageous for various reasons. For instance, because of the extreme size constraints on hearing instruments 102, the batteries of hearing instruments 102 may need to be very small. Maintaining a real time clock in hearing instruments 102 may consume a significant amount of power from a battery or other power source that may be better used for other purposes. Hearing instruments 102 may produce a clock signal that cycles at a given frequency so that hearing instruments 102 is able to track relative time. For instance, hearing instruments 102 may be able to count clock cycles to determine that a given amount of time (e.g., five minutes) has passed following a given clock cycle, but without a real-time clock hearing instruments 102 may not be equipped to relate that relative time to an actual time and date (e.g., 11:34 A.M. on Aug. 22, 2017). Moreover, maintaining a real time clock based on this clock signal may require hearing instruments 102 to continue the clock signal even while hearing instruments 102 is not in use, which may consume a significant amount of battery power.

However, several of the sub-components of the cognitive benefit measure and the body fitness measure are time-dependent. For example, the “use score” sub-component may be based on how much time the wearer of hearing instruments 102 uses the hearing instruments 102 during a scoring period. In another example, the “engagement” sub-component may be based at least in part on how much time the wearer of hearing instruments 102 engages in conversation during a scoring period and how much time the wearer of hearing instruments 102 uses hearing instruments 102 to stream audio media during the scoring period. Moreover, in some examples, computing system 104 may need to determine times associated with log data items received from hearing instruments 102 to determine whether the log data items are associated with a current scoring period.

This disclosure describes techniques that may overcome the problems associated with determining sub-components of the cognitive benefit measure and/or the body fitness measure in the absence of a real-time clock in hearing instruments 102. For example, hearing instruments 102 may maintain a data log that stores log data items, which may include sub-component data. The sub-component data may include data from which values of sub-components may, at least partially, be determined. For example, an inertial measurement unit (IMU) of hearing instruments 102 may periodically write data to the data log indicating the number of steps taken by the wearer of hearing instruments 102. In accordance with one or more techniques of this disclosure, hearing instruments 102 may receive timestamps from a computing device in computing system 104. For example, hearing instruments 102 may receive timestamps from mobile device 106. A timestamp may be a value that indicates a time. For instance, a timestamp may indicate a number of seconds that have passed since a fixed real time (e.g., since Jan. 1, 1970). When recording data (e.g., log data items) to the data log, hearing instruments 102 may include the timestamp in the log data item. For example, hearing instruments 102 may record a log data item in the data log indicating that the wearer of hearing instruments 102 has started using hearing instruments 102. In this example, hearing instruments 102 may include the timestamp in the log data item. In this example, computing system 104 may use this data recorded in the data log to determine the “use score” sub-component.

Thus, computing system 104 may send timestamps to hearing instruments 102. Additionally, computing system 104 may receive a plurality of log data items from hearing instruments 102. Each of the log data items may include log data and one of the timestamps sent to hearing instruments 102 by computing system 104. Computing system 104 may determine, based on the timestamps and the log data in the log data items, at least one of the cognitive benefit measure or the body fitness measure. For instance, computing system 104 may use the timestamps in the log data items to determine which log data items are from a current scoring period and then only use log data in the log data items from the current scoring period when determining values of the sub-components of the cognitive benefit measure and/or body fitness measure.

Hearing instruments 102 may receive timestamps from computing system 104 in response to one or more of various events. For example, hearing instruments 102 may send a timestamp request to computing system 104 when preparing to write data to the data log. In some examples, hearing instruments 102 may periodically request timestamps from computing system 104. In some examples, computing system 104 may be configured to periodically send timestamps to hearing instruments 102 on an asynchronous basis. That is, in this example, it may not be necessary for hearing instruments 102 to send a request to computing system 104 for timestamps. For instance, computing system 104 may send a timestamp to hearing instruments 102 once every 60 seconds, 30 seconds, or other time period. In this example, hearing instruments 102 may store the timestamp (potentially overwriting a previous version of the timestamp) and then include a copy of the timestamp in a log data item when storing the log data item to the data log. Because exact precision may not be necessary when determining values of sub-components of the cognitive benefit measure and the body fitness measure, including an exactly correct time in a log data item may be unnecessary. Thus, the cycle time for hearing instruments 102 receiving timestamps may be set slow enough that an amount of energy consumed by wireless receiving and writing the timestamps to memory may be less than the amount of energy that would be consumed by hearing instruments 102 maintaining its own real time clock, while allowing for reasonable accuracy.

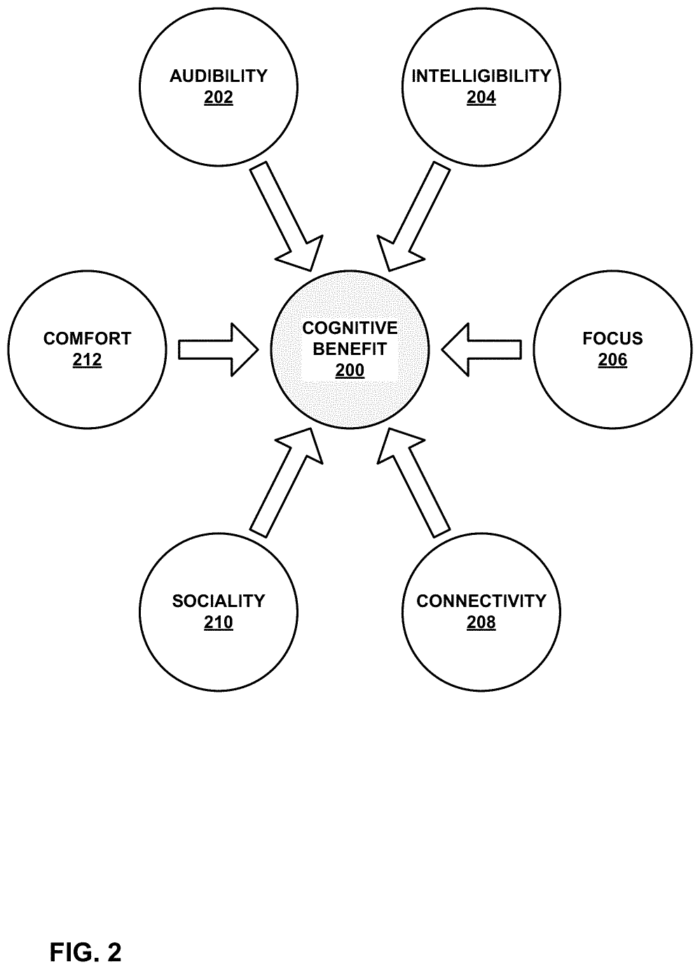

FIG. 2 is a conceptual diagram illustrating contributions of sub-components to a cognitive benefit measure 200, in accordance with one or more aspects of this disclosure. Consistent with the example above, cognitive benefit measure 200 may be determined based on an audibility sub-component 202, an intelligibility sub-component 204, a focus sub-component 206, a connectivity sub-component 208, a sociability sub-component 210, and a comfort sub-component 212. Thus, in the example of FIG. 2, six sub-components (white circles) contribute to the composite cognitive benefit measure (shaded circle); the relationship is denoted by the arrows.

In some examples, computing system 104 may output a graphical user interface (GUI) for display on a display screen. For example, mobile device 106 may output the GUI for display on a display screen of mobile device 106. In another example, server device 108 may generate data defining a webpage comprising the GUI and send the data mobile device 106 or another computing device (e.g., a personal computer) for rendering for display by a web browser application. The GUI may include content similar to that shown in FIG. 2. In response to receiving an indication of user input to select a circle corresponding to a given sub-component, computing system 104 may output for display, information about what is being measured in the given sub-component. However, in some examples, computing system 104 does not cause actual calculations for the sub-components to be displayed. For example, the “sociability” sub-component is a numerical value indicating a measure of the amount of time a wearer of hearing instruments 102 spends in social situations, but the wearer is not privy to the fact that this measure is calculated as the amount of time an automatic environmental classification system of hearing instruments 102 detects speech.

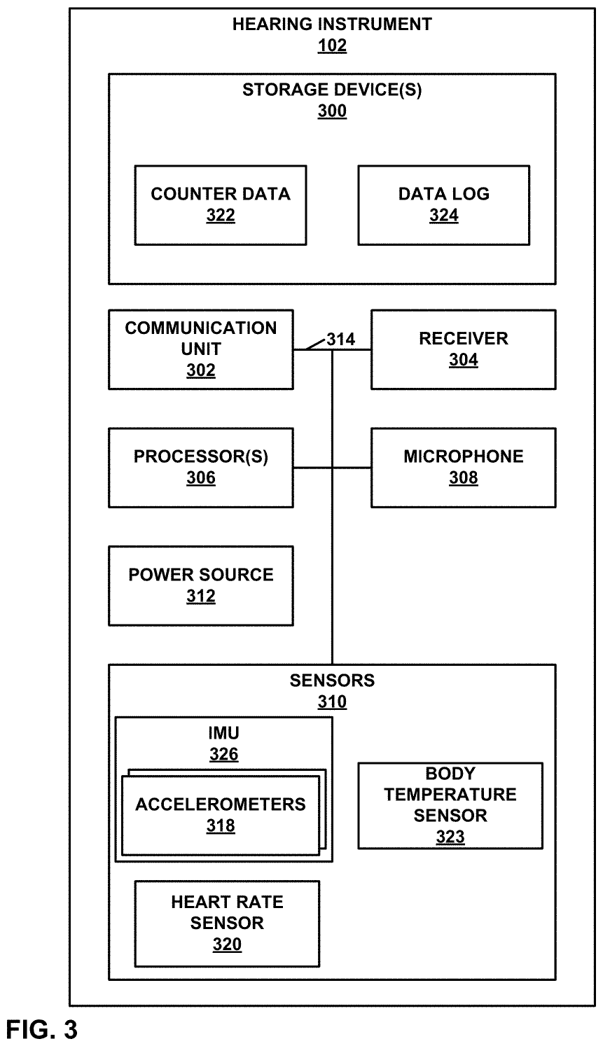

FIG. 3 is a block diagram illustrating example components of hearing instruments 102, in accordance with one or more aspects of this disclosure. In the example of FIG. 3, hearing instruments 102 comprises one or more storage device(s) 300, a radio 302, a receiver 304, one or more processor(s) 306, a microphone 308, a set of sensors 310, a power source 312, and one or more communication channels 314. Communication channels 314 provide communication between storage device(s) 300, radio 302, receiver 304, processor(s) 306, a microphone 308, and sensors 310. Components 300, 302, 304, 306, 308, and 310 may draw electrical power from power source 312, which may be a battery, capacitor, or other type of power source.

In the example of FIG. 3, sensors 310 include one or more accelerometers 318. Furthermore, in examples such as that of FIG. 3, an inertial measurement unit (IMU) 326 includes one or more of accelerometers 318. The IMU 326 may use signals generated by accelerometers 318 for various purposes. For example, the IMU 326 may use the signals generated by accelerometers 318 to count the number of steps that a wearer of hearing assistance device 102 has taken.

Additionally, in the example of FIG. 3, sensors 310 also include a heart rate sensor 320 and a body temperature sensor 323. In other examples, hearing instruments 102 may include more, fewer, or different components. For instance, in other examples, hearing instruments 102 does not include particular sensors shown in the example of FIG. 3. In some examples, heart rate sensor 320 comprises a visible light sensor and/or a pulse oximetry sensor.

Storage device(s) 300 may store data. Storage device(s) 300 may comprise volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 300 may further be configured for long-term storage of information as non-volatile memory space and retain information after power on/off cycles. Examples of non-volatile memory configurations may include magnetic hard discs, optical discs, floppy discs, flash memories, or forms of electrically programmable memories (EPROM) or electrically erasable and programmable (EEPROM) memories.

Radio 302 may enable hearing instrument 102 to send data to and receive data from one or more other computing devices. For example, radio 302 may enable hearing instruments 102 to send data to and receive data from mobile device 106 (FIG. 1). Radio 302 may use one or more of various types of wireless technology to communicate. For instance, radio 302 may use Bluetooth, 3G, 4G, 4G LTE, ZigBee, WiFi, Near-Field Magnetic Induction (NFMI), or another communication technology.

Receiver 304 comprises one or more speakers for generating audible sound. Microphone 308 detects incoming sound and generates an electrical signal (e.g., an analog or digital electrical signal) representing the incoming sound. Processor(s) 306 may process the signal generated by microphone 308 to enhance, amplify, or cancel-out particular channels within the incoming sound. Processor(s) 306 may then cause receiver 304 to generate sound based on the processed signal. In some examples, processor(s) 306 include one or more digital signal processors (DSPs).

Processor(s) 306 may cause radio 302 to transmit one or more of various types of data. For example, processor(s) 306 may cause radio 302 to transmit data to computing system 104. Furthermore, radio 302 may receive audio data from computing system 104 and processor(s) 306 may cause receiver 304 to output sound based on the audio data.

In some examples, hearing instrument 102 is a “plug-n-play” type of device. In some examples, hearing instruments 102 is programmable to help the user manage things like wind noise. Furthermore, in some examples, hearing instruments 102 comprises a custom earmold or a standard receiver module at the end of a RIC cable. The additional volume in a custom earmold may allow room for components such as sensors (accelerometers, heartrate monitors, temp sensors), a woofer-tweeter, (providing richer sound for music aficionados), and an acoustic valve that provides occlusion when desired. In some examples, a six conductor RIC cable is used for in hearing instruments with sensors, woofer-tweeters, and/or acoustic valves.

In the example of FIG. 3, storage device(s) 300 may store counter data 322 and a data log 324. Counter data 322 may include actively updated data used for determining sub-components of a cognitive benefit measure. For example, hearing instruments 102 may store data indicating an amount of time hearing instrument 102 spent streaming media, an amount of time spent in a directional processing mode, and other values. Processor(s) 306 may update counter data 322 at a more frequent rate than data log 324. Processor(s) may flush values from counter data 322 out to data log 324 on a period basis and may reset counter data 322. Additionally, processor(s) 306 may cause radio 302 to send data in data log 324 to computing system 104. For instance, processor(s) 306 may cause radio 302 to send data in data log 324 to computing system 104 in response to radio 302 receiving a request for the data from computing system 104.

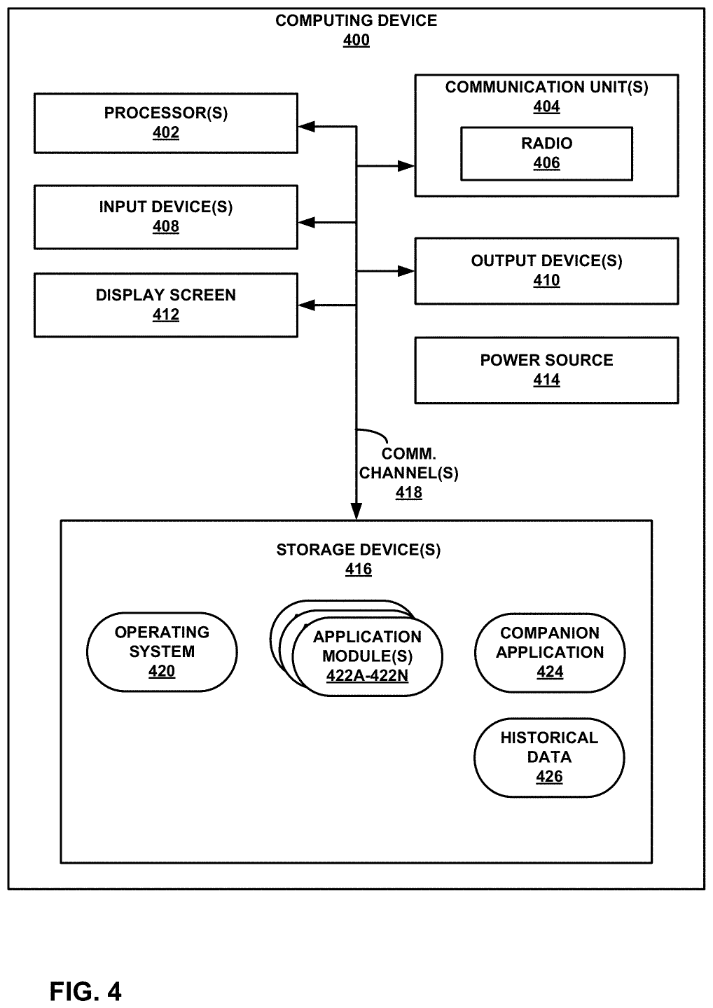

FIG. 4 is a block diagram illustrating example components of computing device 400, in accordance with one or more aspects of this disclosure. FIG. 4 illustrates only one particular example of computing device 400, and many other example configurations of computing device 400 exist. Computing device 400 may be a computing device in computing system 104 (FIG. 1). For instance, computing device 400 may be mobile device 106 or server device 108.

As shown in the example of FIG. 4, computing device 400 includes one or more processors 402, one or more communication units 404, one or more input devices 408, one or more output devices 410, a display screen 412, a power source 414, one or more storage devices 416, and one or more communication channels 418. Computing device 400 may include many other components. For example, computing device 400 may include physical buttons, microphones, speakers, communication ports, and so on. Communication channel(s) 418 may interconnect each of components 402, 404, 408, 410, 412, and 416 for inter-component communications (physically, communicatively, and/or operatively). In some examples, communication channel(s) 418 may include a system bus, a network connection, an inter-process communication data structure, or any other method for communicating data. Power source 414 may provide electrical energy to components 402, 404, 408, 410, 412 and 416.

Storage device(s) 416 may store information required for use during operation of computing device 400. In some examples, storage device(s) 416 have the primary purpose of being a short term and not a long-term computer-readable storage medium. Storage device(s) 416 may be volatile memory and may therefore not retain stored contents if powered off. Storage device(s) 416 may further be configured for long-term storage of information as non-volatile memory space and retain information after power on/off cycles. In some examples, processor(s) 402 on computing device 400 read and may execute instructions stored by storage device(s) 416.

Computing device 400 may include one or more input device(s) 408 that computing device 400 uses to receive user input. Examples of user input include tactile, audio, and video user input. Input device(s) 408 may include presence-sensitive screens, touch-sensitive screens, mice, keyboards, voice responsive systems, microphones or other types of devices for detecting input from a human or machine.

Communication unit(s) 404 may enable computing device 400 to send data to and receive data from one or more other computing devices (e.g., via a communications network, such as a local area network or the Internet). In some examples, communication unit(s) 404 may include wireless transmitters and receivers that enable computing device 400 to communicate wirelessly with the other computing devices. For instance, in the example of FIG. 4, communication unit(s) 404 include a radio 406 that enables computing device 400 to communicate wirelessly with other computing devices, such as hearing instruments 102 (FIG. 1, FIG. 3). Examples of communication unit(s) 404 may include network interface cards, Ethernet cards, optical transceivers, radio frequency transceivers, or other types of devices that are able to send and receive information. Other examples of such communication units may include Bluetooth, 3G, and WI-FI™ radios, Universal Serial Bus (USB) interfaces, etc. Computing device 400 may use communication unit(s) 404 to communicate with one or more hearing instruments (e.g., hearing instruments 102 (FIG. 1, FIG. 3)). Additionally, computing device 400 may use communication unit(s) 404 to communicate with one or more other remote devices (e.g., server device 108 (FIG. 1)).

Output device(s) 410 may generate output. Examples of output include tactile, audio, and video output. Output device(s) 410 may include presence-sensitive screens, sound cards, video graphics adapter cards, speakers, liquid crystal displays (LCD), or other types of devices for generating output.

Processor(s) 402 may read instructions from storage device(s) 416 and may execute instructions stored by storage device(s) 416. Execution of the instructions by processor(s) 402 may configure or cause computing device 400 to provide at least some of the functionality ascribed in this disclosure to computing device 400. As shown in the example of FIG. 4, storage device(s) 416 include computer-readable instructions associated with operating system 420, application modules 422A-422N (collectively, “application modules 422”), and a companion application 424. Additionally, in the example of FIG. 4, storage device(s) 416 may store historical data 426.

Execution of instructions associated with operating system 420 may cause computing device 400 to perform various functions to manage hardware resources of computing device 400 and to provide various common services for other computer programs. Execution of instructions associated with application modules 422 may cause computing device 400 to provide one or more of various applications (e.g., “apps,” operating system applications, etc.). Application modules 422 may provide particular applications, such as text messaging (e.g., SMS) applications, instant messaging applications, email applications, social media applications, text composition applications, and so on.

Execution of instructions associated with companion application 424 may cause computing device 400 to perform one or more of various functions described in this disclosure with respect to computing system 104 (FIG. 1). For example, execution of instructions associated with companion application 424 may cause computing device 400 to configure radio 406 to wirelessly receive data from hearing instruments 102 (FIG. 1; FIG. 3). Additionally, execution of instructions of companion application 424 may cause computing device 400 to determine a cognitive benefit measure, a body fitness measure, and/or a wellness measure for a wearer of hearing instruments 102 and output an indication of the cognitive benefit measure, the body fitness measure, and/or the wellness measure. Although not explicitly recited here for the sake of brevity, instructions associated with companion application 424 may cause computing device 400 to perform one or more of various other actions of computing system 104. In some examples, companion application 424 is an instance of a web application or server application. In some examples, such as examples where computing device 400 is a mobile device, companion application 424 may be a native application.

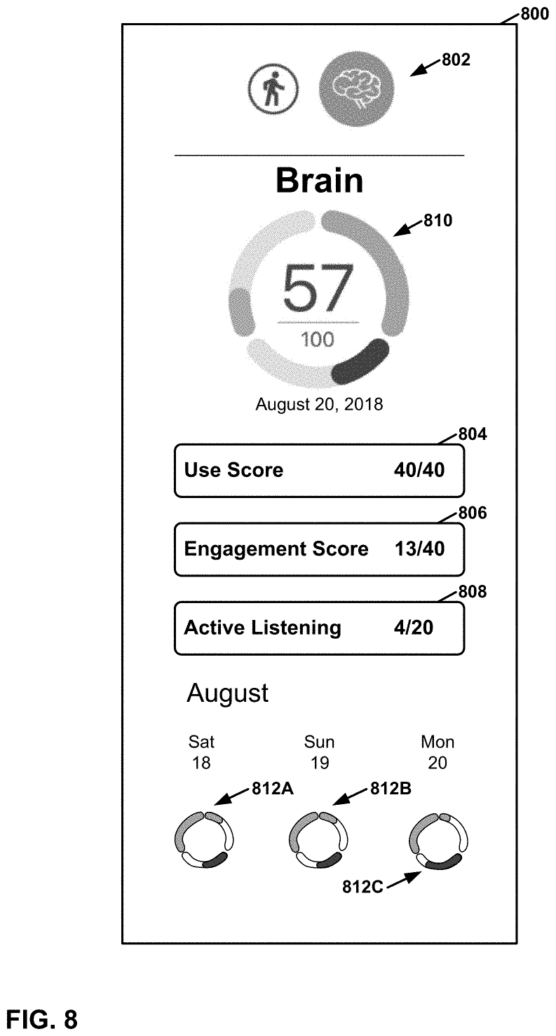

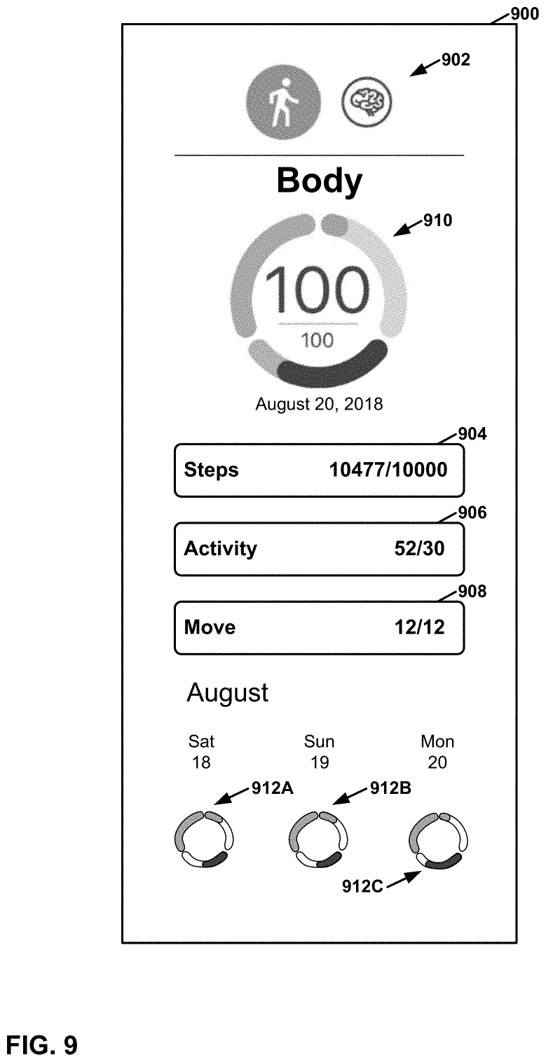

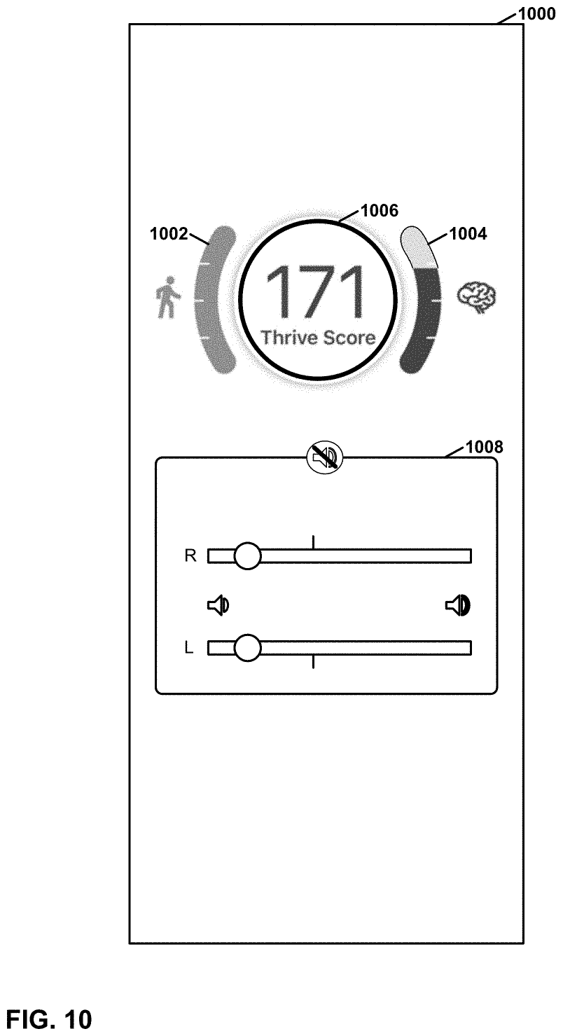

In some examples, a GUI of companion application 424 has a plurality of different sections, that may or may not appear concurrently. For example, the GUI of companion application 424 may include a section for controlling the intensity of sound generated by (e.g., the volume of) hearing instruments 102, a section for controlling how hearing instruments 102 attenuate wind noise, a second for finding hearing instruments 102 if lost, and so on. Additionally, the GUI of companion application 424 may include a cognitive benefit section that displays data regarding a cognitive benefit measure for the wearer of hearing instruments 102. In some examples, the cognitive benefit section of companion application 424 displays a diagram similar to that shown in the example of FIG. 2 or the example of FIG. 8. Additionally, the GUI of companion application 424 may include a body fitness measure section that displays data regarding a body fitness measure for the wearer of hearing instruments 102. In some examples, the body fitness measure section of companion application 424 displays a diagram similar to the example of FIG. 9, described below. The GUI of companion application 424 may also include a wellness measure section that displays data regarding a wellness measure for the wearer of hearing instruments 102. In some examples, the wellness measure section of companion application 424 displays a diagram similar to the example of FIG. 10, described below.

In some examples, companion application 424 may request data for calculating a cognitive benefit measure or body fitness measure from hearing instruments 102 each time mobile device 106 receives an indication of user input to navigate to the cognitive benefit section or body fitness measure section of companion application 424. In this way, a wearer of hearing instruments 102 may get real-time confirmation that companion application 424 is communicating with hearing instruments 102, that the data displayed are current, and may ensure that the wireless transfer of the data-log data does not interrupt or interfere with other processes in companion application 424, or on computing device 400 device. Furthermore, requesting data from hearing instruments 102 only when computing device 400 receives an indication of user input to navigate to the cognitive benefit section, the body fitness measure section, or the wellness measure section of companion application 424 may reduce demands on a power source (e.g., power source 312 of FIG. 3) of hearing instruments 102 (FIG. 1; FIG. 3), relative to computing device 400 requesting the data from hearing instruments 102 on a periodic basis.

Companion application 424 may store one or more of various types of data as historical data 426. Historical data 426 may comprise a database for storing historic data related to cognitive benefit. For example, companion application 424 may store, in historical data 426, cognitive benefit measures, body fitness measures, sub-component values, data from hearing instruments 102, and/or other data. Companion application 424 may retrieve data from historical data 426 to generate a GUI for display of past cognitive benefit measures, body fitness measures, and wellness measures of the wearer of hearing instruments 102.

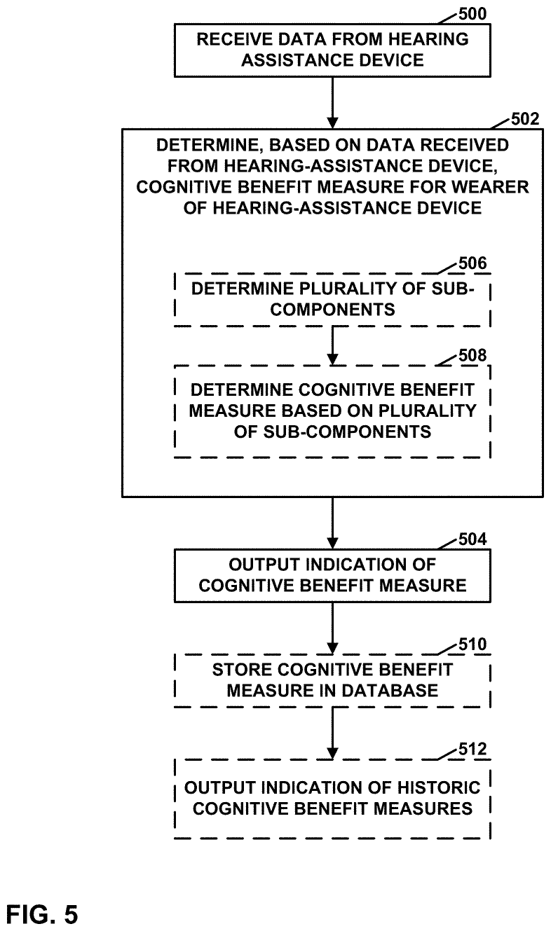

FIG. 5 is a flowchart illustrating an example operation of computing system 104, in accordance with one or more aspects of this disclosure. The flowcharts of this disclosure are provided as examples. In other examples, operations shown in the flowcharts may include more, fewer, or different actions, or actions may be performed in different orders or in parallel.

In the example of FIG. 5, computing system 104 may receive data from hearing instruments 102 (500). For example, computing system 104 may receive data from hearing instruments 102 in response to computing system 104 sending a request for the data to hearing instruments 102. In some examples, computing system 104 receives an indication of user input to access a cognitive benefit section of a GUI of a software application (e.g., companion application 424) running on computing system 104. In this example, in response to receiving the indication of user input, computing system 104 sends a request to hearing instruments 102 and computing system 104 receives the data from hearing instruments 102 in response to the request.

Additionally, computing system 104 may determine, based on the data received from hearing instruments 102, a cognitive benefit measure for a wearer of hearing instruments 102 (502). The cognitive benefit measure may be an indication of a change of a cognitive benefit of the wearer of hearing instruments 102 attributable to use of hearing instruments 102 by the wearer of hearing instruments 102. In some examples, computing system 104 may scale the cognitive benefit measure based on an amount of time the wearer spends wearing hearing instruments 102.

Furthermore, in the example of FIG. 5, computing system 104 may output an indication of the cognitive benefit measure (504). For example, computing system 104 may output a GUI for display that includes a numerical value indicating the cognitive benefit measure. In some examples, to output the indication of the cognitive benefit measures, computing system 104 may send to hearing instruments 102 audio data that represents the cognitive benefit measure in audible form.

As shown in the example of FIG. 5, computing system 104 may, as part of determining the cognitive benefit measure for the wearer of hearing instruments 102, determine a plurality of sub-components of the cognitive benefit measure (506). Additionally, computing system 104 may determine the cognitive benefit measure based on the plurality of sub-components of the cognitive benefit measure (508). For example, computing system 104 may determine a weighted average of the plurality of sub-components to determine the cognitive benefit measure.

As part of determining the plurality of sub-components, computing system 104 may determine an “audibility” sub-component, an “intelligibility” sub-component, a “comfort” sub-component, a “focus” sub-component, a “sociability” sub-component, and a “connectivity” sub-component. For example, computing system 104 may determine an audibility sub-component that is a measure of the improvement in audibility provided to the wearer by hearing instruments 102. For instance, the audibility sub-component may indicate a measure of detected sounds that are amplified sounds. In this example, each of the detected sounds is a sound detected by hearing instruments 102. Furthermore, in this example, each respective amplified sound is a sound that was amplified by hearing instruments 102 because the intensity of the sound was below an audibility threshold of the wearer of hearing instruments 102. In this example, the audibility threshold of the wearer of hearing instruments 102 is an intensity level below which the wearer of hearing instruments 102 is unable to reliably hear the sound.

In some examples, computing system 104 may determine an intelligibility sub-component that indicates a measure of an improvement in speech understanding provided by hearing instruments 102. Furthermore, in some examples, computing system 104 may determine a comfort sub-component that indicates a measure of noise reduction provided by hearing instruments 102. In some examples, computing system 104 may determine a focus sub-component that indicates a measure of time hearing instruments 102 spends in directional processing modes. In this example, each of the respective directional processing modes selectively attenuates off-axis, unwanted sounds. Furthermore, in some examples, computing system 104 may determine a sociability sub-component that indicates a measure of time spent in auditory environments involving speech. In some examples, computing system 104 may determine a connectivity sub-component that indicates a measure of an amount of time hearing instruments 102 spent streaming media from devices connected wirelessly to hearing instruments 102.

In some examples, as part of determining the plurality of sub-components, computing system 104 may determine a “use score” sub-component, an “engagement score” sub-component, and an “active listening” sub-component. In such examples, computing system 104 may determine the cognitive benefit measure (e.g., a brain score) as a weighted sum of the “use score” sub-component, the “engagement score” sub-component, and the “active listening” sub-component. For instance, computing system 104 may determine the cognitive benefit measure such that a first percentage (e.g., 40%) of the cognitive benefit measure is based on the “use score” sub-component, a second percentage (e.g., 40%) of the cognitive benefit measure is based on the “engagement score” sub-component, and a third percentage (e.g., 20%) of the cognitive benefit measure is based on the “active listening” sub-component.

In such examples, the “use score” sub-component may be based on an amount of time during a scoring period that the wearer of hearing instruments 102 has used hearing instruments 102. The wearer of hearing instruments 102 may be considered to be using hearing instruments 102 when hearing instruments 102 is in the wearer's ear and turned on. In some examples, hearing instruments 102 may determine whether hearing instruments 102 are in the wearer's ears based on one or more of various signals generated by sensors 310 (FIG. 3). For instance, hearing instruments 102 may determine whether hearing instruments 102 are in the wearer's ears based on a pattern of motion signals generated by accelerometers 318 (FIG. 3), based on a body temperature signal from body temperature sensor 323, and/or based on a heart rate signal from heart rate sensor 320. In some examples, computing system 104 may determine the “use score” based on a comparison of an in-use time to a time goal. The in-use time may indicate the amount of time that hearing instruments 102 is in the wearer's ear and turned on. The time goal may be a predetermined amount of time (e.g., 12 hours). Thus, in one example, computing system 104 may determine the value of the “use score” sub-component based on the in-use time during the current scoring period divided by the time goal, multiplied by a maximum value of the “use score” subcomponent. Thus, in this example, if the time goal is 12 hours during the current scoring period, the maximum value of the “use score” subcomponent is 40 and the in-use time is 12 hours, computing system 104 may determine that the value of the “use score” sub-component is equal to 40. In some examples, hearing instruments 102 may record log data items in data log 324 that include timestamps of when the wearer started and stopped wearing hearing instruments 102.

The “engagement score” sub-component may be a measure of how much the wearer of hearing instruments 102 participates in activities involving aural engagement during a scoring period. Example types of activities involving aural engagement include engaging in conversation, streaming audio data (e.g., streaming music, streaming audio data from television or a cinema), and other activities that involve the wearer of hearing instruments 102 actively listening to sounds.

In examples where the value of the “engagement score” sub-component is based on the wearer of hearing instruments 102 engaging in conversation, hearing instruments 102 may run an acoustic classifier that classifies sounds detected by hearing instruments 102. For example, the acoustic classifier may determine whether the current sound detected by hearing instruments 102 is silent, speaking and quiet, speaking with noise, music, and wind. In other examples, the acoustic classifier may classify the detected sounds into other categories.

In some examples, computing system 104 may determine the value of the “engagement score” sub-component based at least in part on an amount of time that the sound detected by hearing instruments 102 is classified into a speech category. Hearing instruments 102 may record transitions between categories as log data items in data log 324. In some examples, computing system 104 may determine the value of the “engagement score” sub-component based at least in part of a number of times that hearing instruments 102 determines during the current scoring period that the type of sound detected by hearing instruments 102 transitions to a speech category from another type of sound. For instance, computing system 104 may determine the “engagement score” sub-component based on the progress of the wearer of hearing instruments 102 toward a goal of a particular amount of time that sound detected by hearing instruments 102 is classified into a speech category

Furthermore, in some examples, computing system 104 may determine the “engagement score” sub-component based on multiple activities involving aural engagement. For example, computing system 104 may determine a first component of the “engagement score” sub-component based on engagement in conversation and a second component of the “engagement score” sub-component based on streaming audio data. In some examples, hearing instruments 102 may record log data items in data log 324 that include timestamps of when hearing instruments 102 started and stopped streaming media data. In this example, the first factor may be determined in the same manner as the “sociability” sub-component described elsewhere in this disclosure and the second factor may be determined in the same manner as the “connectivity” sub-component described elsewhere in this disclosure. In this example, a first percentage (e.g., 80%) of the “engagement score” sub-component may be based on the first factor and a second percentage (e.g., 20%) of the “engagement score” sub-component may be based on the second factor. For instance, computing system 104 may determine the “engagement score” as a weighted sum of the first and second factors.

The “active listening” sub-component may be determined based on exposure of the wearer of hearing instruments 102 to a plurality of different acoustic environments during a current scoring period. For example, hearing instruments 102 may determine whether the sound detected by hearing instruments 102 is associated with particular types of acoustic environments. Example types of acoustic environments may include speech, speech with noise, quiet, machine noise, and music. In some examples, hearing instruments 102 may record log data items in data log 324 indicating transitions between acoustic environments and timestamps associated with such transitions. Computing system 104 may increment, based on the log data, the “active listening” sub-component for each different type of acoustic environment that hearing instruments 102 detects during a scoring period. For instance, computing system 104 may increment the “active listening” sub-component by x₁ points (e.g., 4 points) for exposure to a first acoustic environment, x₂ for exposure to a second acoustic environment, and so on, where x₁, x₂, . . . x₄ are the same value or two or more different values. In some examples, computing system 104 may also or alternatively determine the value of the “active listening” sub-component based on progress of the wearer of hearing instruments 102 during the current scoring period toward a goal for the “active listening” sub-component. The goal for the “active listening” sub-component may be an amount of time that hearing instruments 102 spends performing a specified function, such as processing speech, processing sound in a directional mode, etc. In another example, the goal for the “active listening” sub-component may be a number of acoustic environments that the wearer of hearing instruments 102 is to experience during the scoring period.

Furthermore, in some examples, as shown in FIG. 5, computing system 104 may store the cognitive benefit measure in a database (e.g., historical data 426 (FIG. 4)) of historical cognitive benefit measures for the wearer of hearing instruments 102 (510). Additionally, computing system 104 may output an indication of the historical cognitive benefit measures (512). For example, computing system 104 may output a graph for display indicating cognitive benefit measures over time for the wearer of hearing instruments 102. In some examples, the historical information may be based on an hour, day, week, month, and/or year, and may be presented as discrete values or a trend (e.g., graph.). In some examples, to output the indication of the historical cognitive benefit measures, computing system 104 may send to hearing instruments 102 audio data that represents the historical cognitive benefit measures in audible form.

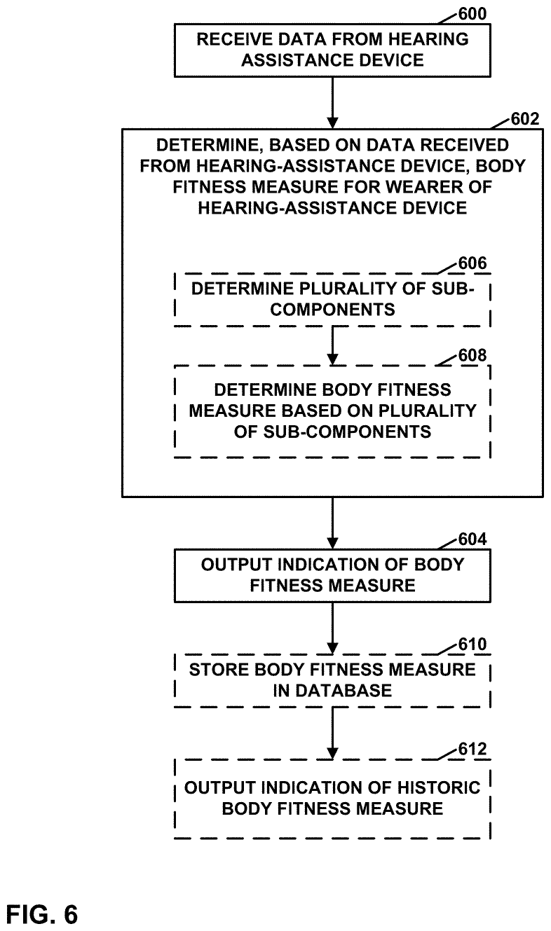

FIG. 6 is a flowchart illustrating an example operation to compute a body fitness measure in accordance with one or more aspects of this disclosure. In the example of FIG. 6, computing system 104 may receive data from hearing instruments 102 (600). For example, computing system 104 may receive data from hearing instruments 102 in response to computing system 104 sending a request for the data to hearing instruments 102. In some examples, computing system 104 receives an indication of user input to access a body fitness measure section of a graphical user interface (GUI) of a software application (e.g., companion application 424) running on computing system 104. In this example, in response to receiving the indication of user input, computing system 104 sends a request to hearing instruments 102 and computing system 104 receives the data from hearing instruments 102 in response to the request. In some examples, computing system 104 may receive a copy of data in data log 324 (FIG. 3) in accordance with any of the examples provided elsewhere in this disclosure.

Additionally, computing system 104 may determine, based on the data received from hearing instruments 102, a body fitness measure for the wearer of hearing instruments 102 (602). The body fitness measure may be an indication of a level of physical activity in which the wearer of hearing instruments 102 has engaged during a scoring period while wearing hearing instruments 102. In some examples, computing system 104 may scale the body fitness measure based on an amount of time the wearer of hearing instruments 102 spends wearing hearing instruments 102.

Furthermore, in the example of FIG. 6, computing system 104 may output an indication of the body fitness measure (604). For example, computing system 104 may output a GUI for display that includes a numerical value indicating the body fitness measure. FIG. 9, described in detail below, is an example GUI for display of the body fitness measure. In some examples, to output the indication of the body fitness measure, computing system 104 may send to hearing instruments 102 audio data that represents the body fitness measure in audible form.

As shown in the example of FIG. 6, computing system 104 may, as part of determining the body fitness measure for the wearer of hearing instruments 102, determine a plurality of sub-components of the body fitness measure (606). Additionally, computing system 104 may determine the body fitness measure based on the plurality of sub-components of the body fitness measure (608). For example, computing system 104 may determine a weighted average of the plurality of sub-components to determine the body fitness measure.

As part of determining the plurality of sub-components, computing system 104 may determine a “steps” sub-component, an “activity” sub-component, and a “move” sub-component. The “steps” sub-component may be based on a number of steps (e.g., while walking or running) that the wearer of hearing instruments 102 has taken during the current scoring period. In some examples, computing system 104 may determine a value of the “steps” sub-component based on the progress during the current scoring period of the wearer of hearing instruments 102 toward a goal for the “steps” sub-component. Furthermore, in some examples, IMU 326 determines the number of steps and hearing instruments 102 writes data indicating the number of steps to data log 324. In some examples, hearing instruments 102 stores timestamps with the number of steps.

The “activity” sub-component may be a measure of vigorous activity in which the wearer of hearing instruments 102 has engaged during the current scoring period. For example, computing system 104 may increment the “activity” sub-component in response to determining that the wearer of hearing instruments 102 has performed a vigorous activity. In some examples, computing system 104 may determine a value of the “activity” sub-component based on the progress during the current scoring period of the wearer of hearing instruments 102 toward meeting a goal for the “activity” sub-component. In such examples, the goal for the “activity” sub-component may be defined as a number of vigorous activities or amount of time engaged in vigorous activities to be performed during the current scoring period.

Computing system 104 or hearing instruments 102 may determine whether the wearer of hearing instruments 102 has performed a vigorous activity in one or more of various ways. For example, computing system 104 or hearing instruments 102 may determine that the wearer of hearing instruments 102 has performed a vigorous activity if computing system 104 has taken more than a given number of steps in a given amount of time. For instance, computing system 104 or hearing instruments 102 may assume that the wearer of hearing instruments 102 has run (or engaged in an activity more vigorous than a brisk walk) if the wearer of hearing instruments 102 has taken more than a threshold number of steps within a given time period.

Hearing instruments 102 may store one or more of various types of data to data log 324 to enable computing system 104 to determine the “activity” sub-component. For example, IMU 326 may output the number of steps taken during a given period. For instance, for every minute, IMU 326 may output the number of steps taken during that minute. Hearing instruments 102 may write a log data item including a timestamp to data log 324 if the number of steps taken during the given period is greater than a threshold associated with vigorous activity.

The “move” sub-component may be based on a number time of intervals during the current scoring period in which the wearer of hearing instruments 102 moves for a given amount of time. For example, computing system 104 may determine the “move” sub-component as a number of hours during a day in which the wearer of hearing instruments 102 was actively moving for more than 1 minute. In some examples, computing system 104 may determine the “move” sub-component based on progress of the wearer of hearing instruments 102 during the current scoring period toward a goal for the “move” sub-component. In such examples, the goal for the “move” sub-component may be defined as a given number of time intervals during the current scoring period in which the wearer of hearing instruments 102 moves for the given amount of time.

Hearing instruments 102 may store one or more of various types of data to data log 324 to enable computing system 104 to determine the “move” sub-component. For instance, in one example, hearing instruments 102 may receive timestamps from computing system 104 as described elsewhere in this disclosure. Furthermore, in this example, hearing instruments 102 may write data to data log 324 indicating that the wearer has started moving with a first timestamp and data indicating that the wearer has stopped moving with a second timestamp. Computing system 104 may analyze such data to determine whether the wearer of hearing instruments 102 was active for the given amount of time during a time interval.

Furthermore, in some examples, as shown in FIG. 6, computing system 104 may store the body fitness measure in a database (e.g., historical data 426 (FIG. 4)) of historical body fitness measures for the wearer of hearing instruments 102 (610). Additionally, computing system 104 may output an indication of the historical body fitness measures (612). For example, computing system 104 may output a graph for display indicating body fitness measures over time for the wearer of hearing instruments 102. In some examples, the historical information may be based on an hour, day, week, month, or year, and may be presented as discrete values or a trend (e.g., graph.). In some examples, to output the indication of the historical body fitness measures, computing system 104 may send to hearing instruments 102 audio data that represents the historical body fitness measures in audible form.

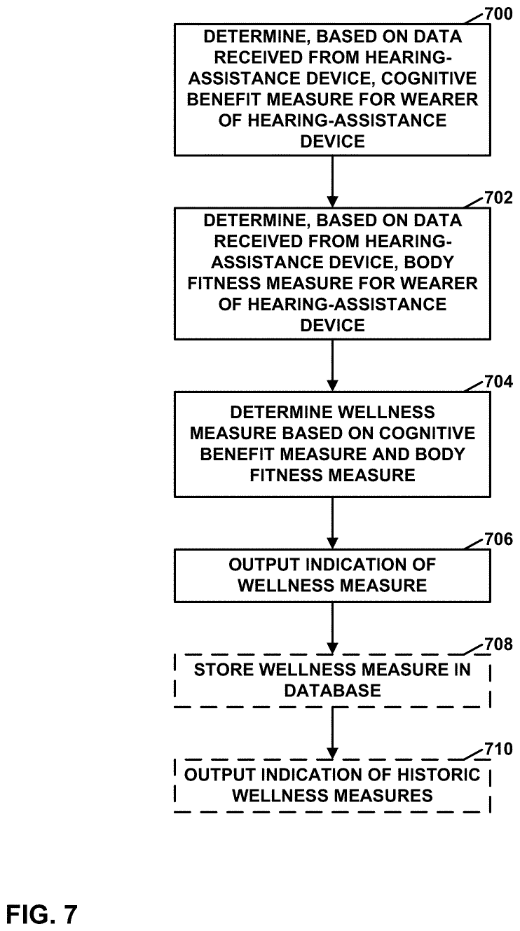

FIG. 7 is a flowchart illustrating an example operation to compute a wellness measure in accordance with one or more aspects of this disclosure. In the example of FIG. 7, computing system 104 may determine, based on the data received from hearing instruments 102, a cognitive benefit measure for a wearer of hearing instruments 102 (700). Computing system 104 may determine the cognitive benefit measure in the manner described above with respect to FIG. 5.

Furthermore, in the example of FIG. 7, computing system 104 may determine, based on the data received from hearing instruments 102, a body fitness measure for the wearer of hearing instruments 102 (702). Computing system 104 may determine the body fitness measure in the manner described above with respect to FIG. 6.

Computing system 104 may determine a wellness measure based on the cognitive benefit measure for the wearer of hearing instruments 102 and the body fitness measure for the wearer of hearing instruments 102 (704). In various examples, computing system 104 may determine the wellness measure in various ways. For example, computing system 104 may determine the wellness measure as a weighted sum of the cognitive benefit measure and the body fitness measure. For instance, in this example, computing system 104 may determine the wellness measure with equal weightings, e.g., a 50% weighting to the cognitive benefit measure and 50% weighting to the body fitness measure. In other examples, computing system 104 may use unbalanced (i.e., different) weightings of the cognitive benefit measure and the body fitness measure. The weighting for the cognitive benefit measure may be greater than the weighting for the body fitness measure. Alternatively, the weighting for the cognitive benefit measure may be less than the weighting for the body fitness measure.

As one example, computing system 104 may determine the wellness measure with a 60% weighting to the cognitive benefit measure and a 40% weighting to the body fitness measure.

In the example of FIG. 7, computing system 104 may output an indication of the wellness measure (706). For example, computing system 104 may output a GUI for display that includes a numerical value indicating the wellness measure. FIG. 10, described in detail below, is an example GUI for display of the wellness measure. In some examples, to output the indication of the wellness measure, computing system 104 may send to hearing instruments 102 audio data that represents the wellness measure in audible form.

Furthermore, in some examples, as shown in FIG. 7, computing system 104 may store the wellness measure in a database (e.g., historical data 426 (FIG. 4)) of historical wellness measures for the wearer of hearing instruments 102 (708). Additionally, computing system 104 may output an indication of the historical wellness measures (710). For example, computing system 104 may output a graph for display indicating wellness measures over time for the wearer of hearing instruments 102. In some examples, the historical information may be based on an hour, day, week, month, or year, and may be presented as discrete values or a trend (e.g., graph.). In some examples, to output the indication of the historical wellness measures, computing system 104 may send to hearing instruments 102 audio data that represents the historical wellness measures in audible form.

FIG. 8 is an example GUI 800 for display of a cognitive benefit measure in accordance with one or more aspects of this disclosure. In the example of FIG. 8, GUI 800 includes controls 802 that allow a user to switch between a user interface for display of the cognitive benefit measure and a user interface for display of a body fitness measure.

In the example of FIG. 8, the cognitive benefit measure is based on a use score sub-component, an engagement score sub-component, and an active listening sub-component. Computing system 104 may determine the use score sub-component, the engagement score sub-component, and the active listening sub-component in the manner described in any of the examples provided elsewhere in this disclosure. Feature 804 of GUI 800 indicates a value of the use score sub-component. Feature 806 of GUI 800 indicates a value of the engagement score sub-component. Feature 808 of GUI 800 indicates a value of the active listening sub-component. In features 804, 806, and 808 of GUI 800, the value before the “/” mark indicates a current value of the sub-component and the value after the “/” mark indicates a goal for the sub-component.

Furthermore, in the example of FIG. 8, GUI 800 includes a circular diagram 810 having segments corresponding to the sub-components of the cognitive benefit measure. Each of the segments is filled in an amount proportional to the wearer's progress toward meeting the goals for the sub-components of the cognitive benefit measure. Additionally, circular diagram 810 may include a numerical value indicating the wearer's cognitive benefit measure (e.g., 57 in the example of FIG. 8) and a numerical value indicating the wearer's cognitive benefit measure goal for the cognitive benefit measure (e.g., 100 in the example of FIG. 8). The wearer's cognitive benefit measure goal is the wearer's goal for the cognitive benefit measure.

GUI 800 also includes historical icons 812A, 812B, and 812C (collectively, “historical icons 812”). In the example of FIG. 8, like circular diagram 810, historical icons 812 include segments with filled portions corresponding to indicate the wearer's progress toward meeting the goals for the sub-components on previous days, e.g., Saturday, Sunday and Monday in the example of FIG. 8. In response to receiving an indication of user selection of one of historical icons 812, computing system 104 may output for display a GUI having more details regarding the wearer's cognitive benefit measure for the day corresponding to the selected historical icon.

FIG. 9 is an example GUI 900 for display of a body fitness measure in accordance with one or more aspects of this disclosure. In the example of FIG. 9, GUI 900 includes controls 902 that allow a user to switch between a user interface for display of the cognitive benefit measure and a user interface for display of a body fitness measure.

In the example of FIG. 9, the body fitness measure is based on a steps sub-component, an activity sub-component, and a movement sub-component. Computing system 104 may determine the steps sub-component, the activity sub-component, and the movement sub-component in the manner described in any of the examples provided elsewhere in this disclosure. Feature 904 of GUI 900 indicates a value of the steps sub-component. Feature 906 of GUI 900 indicates a value of the activity sub-component. Feature 908 of GUI 900 indicates a value of the movement sub-component. In features 904, 906, and 908 of GUI 900, the value before the “/” mark indicates a current value of the sub-component and the value after the “/” mark indicates a goal for the sub-component.

Furthermore, in the example of FIG. 9, GUI 900 includes a circular diagram 910 having segments corresponding to the sub-components of the body fitness measure. Each of the segments is filled in an amount proportional to the wearer's progress toward meeting the goals for the sub-components of the body fitness measure. Additionally, circular diagram 910 may include a numerical value indicating the wearer's body fitness measure (e.g., 100 in the example of FIG. 9) and a numerical value indicating the wearer's body fitness measure goal (e.g., 100 in the example of FIG. 9).

GUI 900 also includes historical icons 912A, 912B, and 912C (collectively, “historical icons 912”). Like circular diagram 910, historical icons 912 include segments with filled portions corresponding to the wearer's progress toward meeting the goals for the sub-components on previous days, e.g., Saturday, Sunday and Monday in the example of FIG. 9. In response to receiving an indication of user selection of one of historical icons 912, computing system 104 may output for display a GUI having more details regarding the wearer's cognitive benefit measure for the day corresponding to the selected historical circular diagram.

FIG. 10 is an example GUI 1000 for display of a wellness measure in accordance with one or more aspects of this disclosure. In the example of FIG. 10, GUI 1000 includes a body fitness measure feature 1002 and a cognitive benefit measure feature 1004. Body fitness measure feature 1002 is filled in an amount proportional to the wearer's progress toward meeting the wearer's body fitness measure goal. Cognitive benefit measure feature 1004 is filled in an amount proportional to the wearer's progress toward meeting the wearer's cognitive benefit measure goal. The wearer's cognitive benefit measure goal may also be referred to herein as the cognitive benefit measure goal. Additionally, in the example of FIG. 10, GUI 1000 includes a wellness measure feature 1006 (e.g., indicated by “Thrive Score” in FIG. 10) that includes a numeric value indicating the wearer's wellness measure.

In the example of FIG. 10, GUI 1000 may be a primary screen of companion application 424. Because controlling the volume of hearing instrument(s) may be the feature for which the user uses companion application 424 the most, GUI 1000 may be designed to indicate the wearer's wellness measure along with volume controls 1008 in order to bring the wearer's wellness measure to the user's attention.

In some examples, a computing system (e.g., computing system 104, hearing instruments 102, and/or one or more other devices) may detect one or more user behavior conditions using hearing instruments 102. The computing system may comprise one or more processors. The user behavior conditions may be measures of behavior of the wearer of hearing instruments 102. In this example, the computing system may determine a wellness measure based on the one or more conditions.

The user behavior conditions may include the cognitive benefit measure, the body fitness measure, or other measures of the behavior of the wearer of hearing instruments 102. For instance, the cognitive benefit measure may be considered a measure of user behavior with respect to how the wearer of hearing instruments 102 uses hearing instruments 102. Similarly, the body fitness measure may be considered a measure of user behavior with respect to physical activity behavior in which the wearer of hearing instruments 102 engages. Thus, detecting one or more user behavior conditions may include detecting activity information (e.g., the body fitness measure) and detecting hearing information (e.g., the cognitive benefit measure). For instance, the computing system may determine a cognitive measure and a body measure. The computing system may further determine the wellness measure using the cognitive measure and the body measure. In some such examples, one or more of hearing instruments 102 determine the wellness measure.

In some examples, the computing system may determine the wellness measure based at least in part on the activity information and the hearing information. In some examples, the hearing information includes one or more of hearing aid usage, user engagement, and active listening. In some examples, information relating to the user behavior conditions may be transmitted (e.g., wirelessly or non-wirelessly) from the hearing instrument to a computing device (e.g., a computing device in computing system 104) and the computing device determines the wellness measure using the transmitted information.

Some examples of this disclosure include measures of use of a heart rate monitor, scoring measures of cardiovascular fitness, or other physiological parameters, to the examples provided elsewhere in this disclosure for measuring physical fitness (Body Score), cognitive wellness (Brain Score), and the combination of the two in a measure of overall wellness (Thrive Score).

This disclosure describes techniques that may integrate cardiovascular data into a measure of physical wellness or into a measure of overall wellness. For instance, an optical sensor (e.g., of one or more of hearing instruments 102) may be used to measure heart rate, collect data related to heart rate, or other data. The techniques of this disclosure may be applied to other physiological data collected by this or other sensors.

An optical sensor in a hearing instrument is capable of measuring the heart rate of a user (among other things) by interpreting changes in the reflectance from the vasculature of the ear. The following describes one technique for increasing a point total of a user for using the optical sensor or other types of sensors to monitor their cardiovascular health.

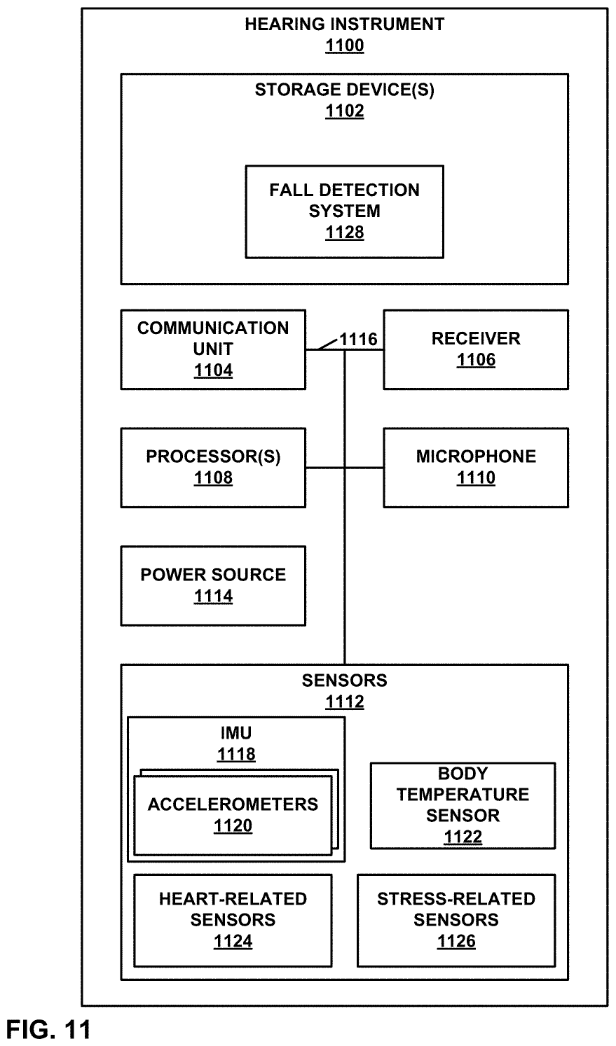

FIG. 11 is a block diagram illustrating example components of a hearing instrument 1100, in accordance with one or more aspects of the disclosure. In the example of FIG. 11, hearing instrument 1100 includes storage devices 1102, communication unit 1104, receiver 1106, processor(s) 1108, microphone 1110, sensors 1112, power source 1114, and communication channels 1116. Sensors 1112 may include IMU 1118 (which may include one or more accelerometers 1120) and body temperature sensors 1122. Hearing instrument 1100 and these components of hearing instrument 1100 may be implemented in the manner described with respect to corresponding parts of FIG. 3.

In the example of FIG. 11, sensors 1112 additionally include heart-related sensors 1124 and stress-related sensors 1126. In other examples, sensors 1112 of hearing instrument 1100 may include more, fewer, or different sensors. For instance, in one example, sensors 1112 do not include one or more of IMU 1118, body temperature sensor 1122, heart-related sensors 1124, or stress-related sensors 1126.

Heart-related sensors 1124 may include one or more photoplethysmography (PPG) sensors, electrodes for collecting ECG signals, and/or other types of sensors that generate heart-related information (i.e., heart-related data). Heart-related data includes data related to a user's heart. Stress-related sensors 1126 may include one or more respiration sensors, electrodes for collecting EEG signals, and/or other types of sensors that generate information that may be used to determine an emotional stress level of the user of hearing instruments 102 (i.e., stress-related information/data).

In some of the examples provided elsewhere in this disclosure, the measure of physical wellness (the “Body Score”) has three components: (1) Steps, (2) Activity, and (3) Move. In some examples, a fourth component to the Body Score (which may be referred to as “Heart,” “Heart Health,” or some other name) may be included in the measure of physical wellness. In some examples, the Heart component tracks how many times a user of hearing instruments 102 uses the heart rate measurement feature of hearing instruments 102.

In this example, the computing system may increase a point total of the user of hearing instruments 102 for checking various measures of their cardiovascular health. In some examples, there are two measures of cardiovascular health, but the system of this disclosure for rewarding health monitoring may apply to other measures as well. The first measure is a heart rate (HR) “on demand,” in which the user explicitly checks their heart using a mobile application (e.g., companion application 424 of FIG. 4) as a user interface, and one or more of heart-related sensors 1124 (e.g., an embedded optical sensor in one or more of hearing instruments 102) is used as measurement tools. Thus, the computing system may determine the heart rate of the user based on signals generated by heart-related sensors 1124. For instance, in an example where heart-related sensors 1124 include a PPG sensor, the computing system may determine the heart rate of the user based on changes to light transmitted or reflected off the user's skin to a photodiode of the PPG sensor. The user of hearing instruments 102 may initiate a heart rate measurement while at rest, or while engaging in any number of activities such as exercise. Computing system 104 (or hearing instruments 102) may add points to e.g., a “Heart Health” subcategory of the Body Score, and thus may add the points to a composite Body Score and/or a composite Thrive Score.

In some examples, a second measure of cardiovascular health is Heart Rate Recovery (HRR). The Heart Rate Recovery feature may guide a user through a step-by-step routine that measures how quickly their heart rate returns to normal after exercise, which is an established measure of cardiovascular fitness. The faster the user's heart rate returns to normal, the stronger and healthier the user's heart. The user may explicitly initiate the Heart Rate Recovery routine and the computing system may increase a point total of the user for the completion of the Heart Rate Recovery routine. Computing system 104 (or hearing instruments 102) may add points to the “Heart Health” subcategory of the Body Score, and thus may the points to the composite Body Score and/or the composite Thrive Score.

Features similar to features 904, 906, and 908 (FIG. 9) may be included for the “Heart Health” subcategory. Similarly, in the example of FIG. 9, ring segments may be included in circular diagram 910 and historical icons 912 for the “Heart Health” subcategory.

In some example implementations of the Body Score, the computing system may increase a point total of the user by a maximum of a (e.g., 40) points for completing the goal set for number of steps (“Steps”), the computing system may increase the point total of the user by a maximum of b (e.g., 40) points for completing the goal set for the number of minutes of activity beyond a brisk walk (“Activity”), and the computing system may increase the point total of the user by a maximum of c (e.g., 20) points for completing the goal of moving from rest a certain number of times per hour per day (“Move”), where a, b, and c are positive numbers. These points total d (e.g., 100) possible points obtained to reach the maximum Body Score for one day. In one example of this disclosure, the maximum number of points that the computing system may add to the point total for the user in one day for “Steps” would remain at a, the maximum number of points awarded for “Activity” would decrease (e.g., to 20), the maximum number of points awarded for “Move” would remain at c, and the remaining points (e.g., 20 points) from the “Activity” subcomponent of the Body Score may be allocated to the “Heart Health” subcomponent.

In some examples of this disclosure, the computing system (e.g., computing system 104, hearing instruments 102, or another device) may reward the user of hearing instruments a given number of points (e.g., 2 points) each time the user measures their heart rate on demand, up to a given number of measures (e.g., 6 measures) per day, for a maximum point allowance of a given number of points (e.g., 12 points) per day for measuring their heart rate. The computing system may reward the user of hearing instruments 102 with a given number of points (e.g., 4 points) each time the user completed the Heart Rate Recovery routine, up to a given number of measures (e.g., 2 measures) per day, for a maximum point allowance of a given number of points (e.g., 8 points) per day for measuring their heart rate recovery. The maximum point allowance per day between these two measures of cardiovascular health would be a particular number of points (e.g., 20 points). The points earned in the Heart Health subcomponent of the Body Score (e.g., ranging from 0-20), may be added to the composite Body Score, and the composite Thrive Score. Other reward structures may be applied, as are any number of different measures of cardiovascular health.

In some examples, the Heart Health component is included in the Body Score, and the computing system increases the point total of the user of hearing instruments 102 for meeting their cardiovascular goals. Example goals may include one or more of: (1) achieving a resting heart rate within a range, (2) achieving an active heart rate (i.e., heart rate during exercise) within a range of target elevated heart rates, (3) elevating heart rate to within a range of target elevated heart rates a certain number of times per day, (4) achieving a Heart Rate Recovery score that is within a target range of scores, (5) achieving a Heart Rate Recovery score that is lower than an accumulating average of scores, thus indicating an improvement in heart health over time, or (6) achieving a Heart Rate Recovery score that is some percentage lower than measures taken previously. Of course, any number of other cardiovascular goals are possible. The values that define all of these goals, both specified and unspecified, may be set by the user of hearing instruments 102, their physician or caregiver, a loved one such as a family member, or some other third-party user.

In some examples, the computing system adds bonus points to the Body score when the user of hearing instruments 102 measures their heart rate. In such examples, a separate component for heart health is not included in the Body Score. In such examples, the existing point structure of the Body score remains unchanged. The computing system may add points on top of the Body Score (e.g., as calculated in examples provided elsewhere in this disclosure) such that the user has more ways to achieve a perfect score. The maximum Body Score (e.g., of 100) does not change. For instance, in one such example, the computing system may increase a point total of the user with 2 points per cardiovascular measure taken during a single day, whether heart rate on demand or Heart Rate Recovery, up to a maximum of 10 bonus points per day.

In some examples, the computing system adds bonus points to the aggregate Thrive score when the user of hearing instruments 102 measures their heart rate. In such examples, neither the Body Score nor Thrive Score includes a separate component for heart health. In such examples, the existing point structure of the Body score (e.g., as calculated in examples provided elsewhere in this disclosure) may remain unchanged. For instance, there may be 100 possible points for the Brain Score, 100 possible points for the Body Score, and 200 possible points for the Thrive Score. In such examples, the computing system may add points on top of the existing Thrive Score such that the user has more ways to achieve a perfect score. For instance, in one such example, the computing system may add 2 points per cardiovascular measure taken during a single day, whether heart rate on demand or Heart Rate Recovery, up to a maximum of a given number (e.g., 10) bonus points per day.

In some examples, a third component is included in the Thrive Score. In such examples, a modified Thrive Score is composed of the existing Brain and Body Scores, plus a Heart Health Score. In one such example, the user of hearing instruments 102 is rewarded for measuring their cardiovascular health. The rewards may be use-based or may be goal-based, as described elsewhere in this disclosure. In other words, the computing system may increase a point total of the user using features the heart rate measurement feature or heart rate recovery feature; or the computing system may increase the point total of the user for making progress toward a goal. In some examples where a third component for heart health is included in the Thrive Score, a Heart Health Score may be worth a maximum of 100 points per day, thus making the Thrive Score worth a maximum of 300 points per day.

While techniques of this disclosure may be implemented in an application (e.g., companion application 424 of FIG. 4), it will be recognized that the techniques of this disclosure may be implemented on many different devices that can be connected (wired or wirelessly) to one or more of hearing instruments 102. In some examples, the techniques of this disclosure may be implemented in software for fitting hearing instruments 102, where such software runs on a computer which may be one such “device.”

While this disclosure describes measures of heart rate and heart rate recovery, the techniques of this disclosure may be applied to different measures of cardiovascular fitness, such as the following:
Resting heart rate (classified by the patient, or by the IMU of the hearing device)
Elevated heart rate (during generic exercise, or activity classified by hearing device)
Heart rate variability
Blood pressure
Blood oxygenation

While this disclosure describes techniques for using cardiovascular data, or the act of collecting cardiovascular data, to augment a measure of physical wellness, the same type of augmentation of the wellness measure could be accomplished using different sensors, and different physiological measures, such as the following:
Electrocardiogram (ECG) data
Electroencephalogram (EEG) data
Blood glucose data
Body temperature data
Body hydration level data
Respiratory data

Thus, some examples of this disclosure may relate to scoring of (e.g., increasing a point total for) biometric data using hearing instruments and a user interface, scoring of (increasing a point total for) cardiovascular data using hearing device and mobile user interface, and/or automatic classification of activity of the user of hearing instruments 102 when a heart rate measurement is made, thus providing context for each measurement.

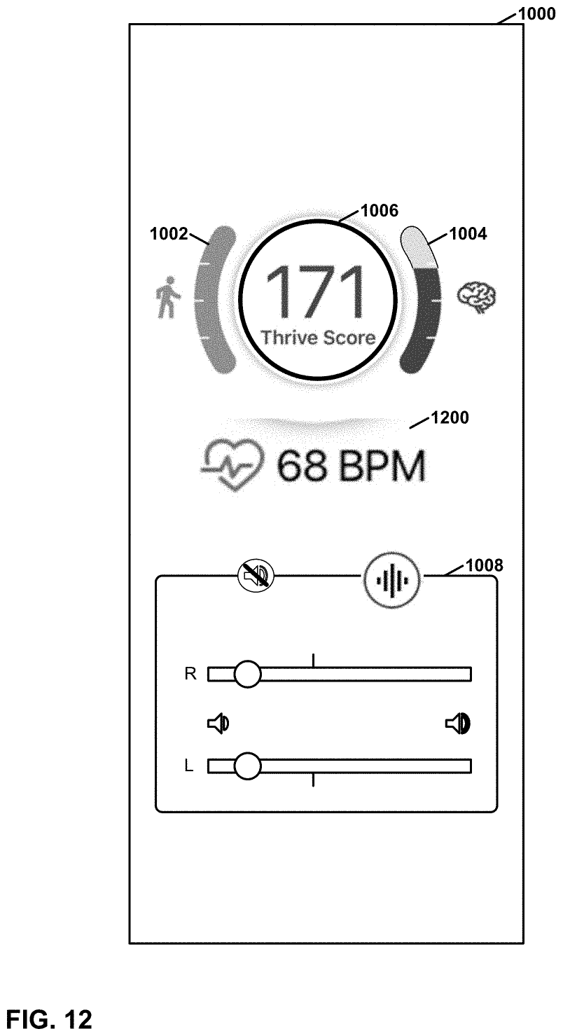

FIG. 12 is an example GUI for display of a wellness measure in accordance with one or more aspects of this disclosure. In the example of FIG. 12, GUI 1000 further includes a heart rate element 1200 indicating a most recent heart rate measure. In some examples, heart rate element 1200 does not indicate a live measurement of the user's heart rate, but rather indicates a heart rate as recorded during a most recent time the user initiated a heart rate measurement process. Not tracking the user's current heart rate may save electrical energy and may avoid depleting a power source of hearing instruments 102. In other examples, heart rate element 1200 indicates a live (e.g., current) heart rate of the user.

In some examples, in response to receiving an indication of user input to select heart rate element 1200, a computing system (e.g., computing system 104, hearing instruments 102, and/or one or more other devices) may display a heart health interface.

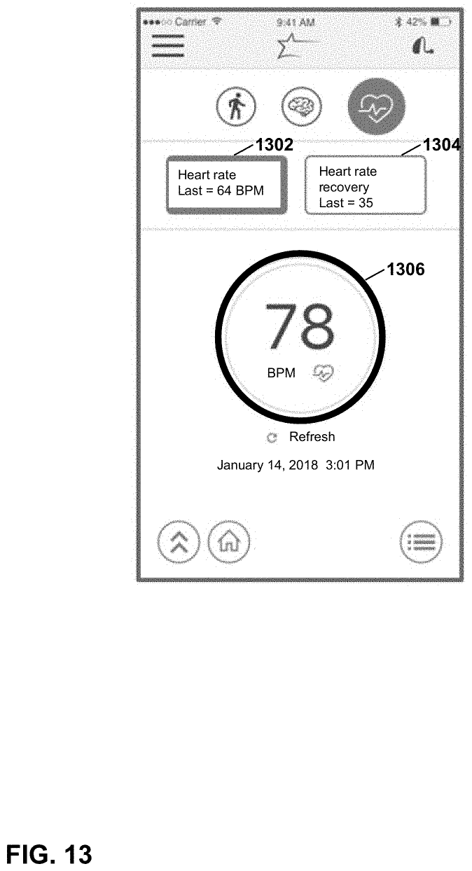

FIG. 13 is an example GUI for display of a heart health interface 1300 in accordance with one or more aspects of this disclosure. In the example of FIG. 13, heart health interface 1300 includes a feature indicating a most recent heart rate 1302 and a feature 1304 indicating a score for heart rate recovery. In response to receiving an indication of user input to select feature 1304, the computing system may begin a guided routine to assess the heart rate recovery of the user of hearing instruments 102.

Additionally, in the example of FIG. 13, heart health interface 1300 may include a feature 1306. In response to receiving an indication of user input to select feature 1306, the computing system may initiate a measurement of the heart rate of the user of hearing instruments 102.

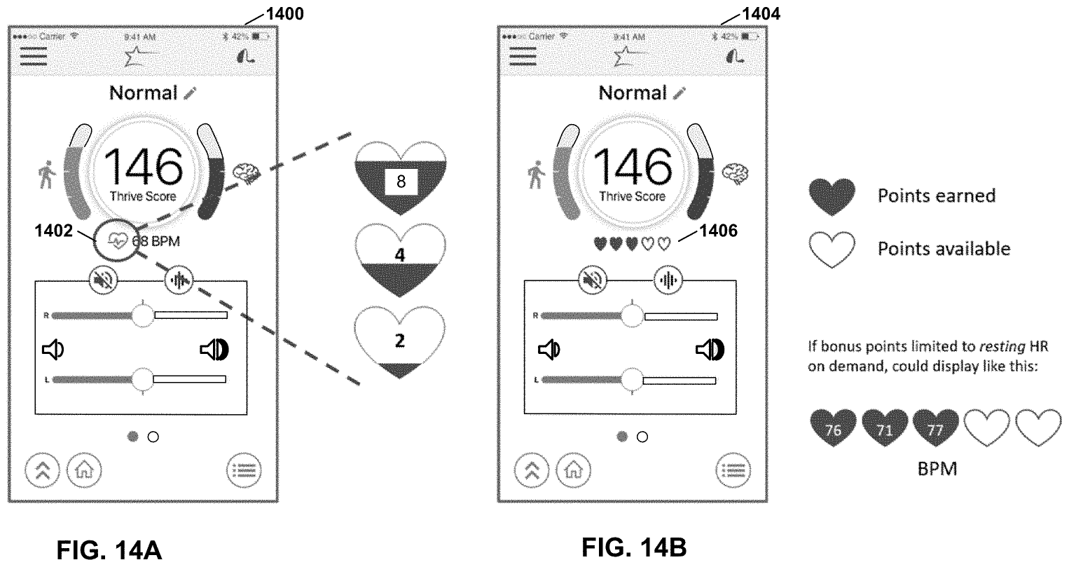

FIG. 14A is a conceptual diagram illustrating a variant of the GUI of FIG. 12, in accordance with one or more aspects of this disclosure. In the example of FIG. 14A, a GUI 1400 may include a heart icon 1402. Heart icon 1402 may show various levels of fullness depending on a number of heart health points earned. In some examples, heart icon 1402 may include text indicating the number of heart health points earned.

FIG. 14B is a conceptual diagram illustrating a variant of the GUI of FIG. 12, in accordance with one or more aspects of this disclosure. In the example of FIG. 14B, heart rate element 1200 is replaced in GUI 1404 with fillable heart icons 1406. Fillable heart icons 1406 may be filled in accordance with heart health points earned. For example, fillable heart icons 1406 may be filled in response to the user initiating a measurement of the user's resting heart rate. Furthermore, as shown in the example of FIG. 14B, fillable heart icons 1406 may include text indicating the users' resting heart rate during corresponding heart rate measurement sessions.

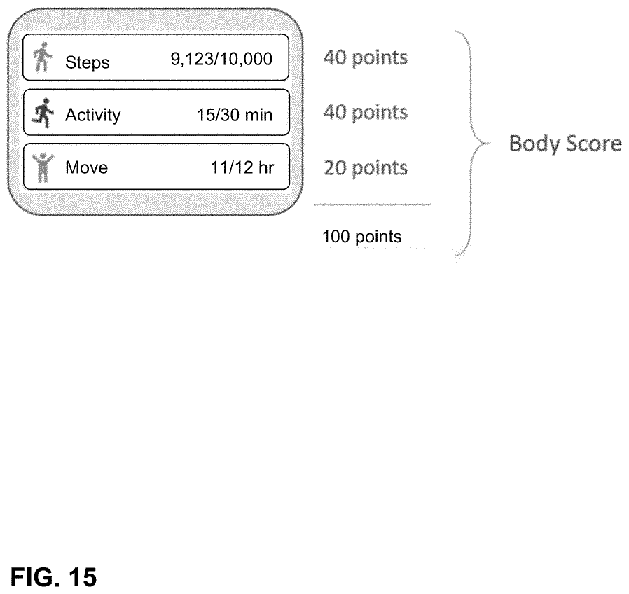

FIG. 15 is a conceptual diagram illustrating an example calculation technique for a body score, in accordance with one or more aspects of this disclosure. In the example of FIG. 15, a body score may have a steps sub-component, an activity sub-component, and a move sub-component, as described elsewhere in this disclosure. In the example of FIG. 15, the steps sub-component has a maximum of 40 points, the activity sub-component has a maximum of 40 points, and the move sub-component has a maximum of 20 points, for a total of 100 points. In this example, bonus points may be added to the body score when the user of hearing instruments 102 measures their heart rate. In the example of FIG. 15, the points structure of the body score is not changed by such bonus points. This may give the user of hearing instruments 102 more ways to get a perfect score. In some examples, the content of FIG. 15 may form part of a GUI, such as GUI 1000 (FIG. 11), GUI 1400 (FIG. 14A), GUI 1404 (FIG. 14B) and/or other GUIs.

In some instances, the scoring structure of FIG. 15 with bonus points may be used with GUI 1400 (FIG. 14A) or GUI 1404 (FIG. 14B). In some such examples, the bonus points may be unclassified. “Unclassified” points may be earned by taking any heart health measure; it would be up to the user to determine which measures to take, rather than needing some from this category, some from that category, to earn all available points.

Such unclassified bonus points may provide credit for a resting heart rate, elevated heart rate, and using the heart rate recovery routine. In some examples, the bonus points may be classified. “Classified” points may be separated into different measures of heart health, such as resting heart rate or heart rate recovery. In order to earn full credit, a variety of different measures would have to be made (as opposed to just resting heart rate, for example). Such classified bonus points may be for checking resting heart rate. In some such examples, there may be 2 points per measure, with a maximum of 10 bonus points.

In some examples, to provide credit for resting heart rate, elevated heart rate, and using the heart rate recovery routine, boundaries may be set for what constitutes a range of desired values for those metrics. Those boundaries could be set by a manufacturer of hearing instruments 102, by the patient alone, or in conjunction with a medical professional. For example, there are normative data for what is deemed to constitute a healthy resting heart rate, or healthy heart rate recovery value. The computing system may award users for having measurement values that are deemed healthy. The measurement values that are deemed healthy may be defined as a function of the age of the user. For users who have heart health values outside of the healthy, desired range, the computing system may encourage the users to work towards betterment of their heart health, or encourage the users to consult with a medical professional.

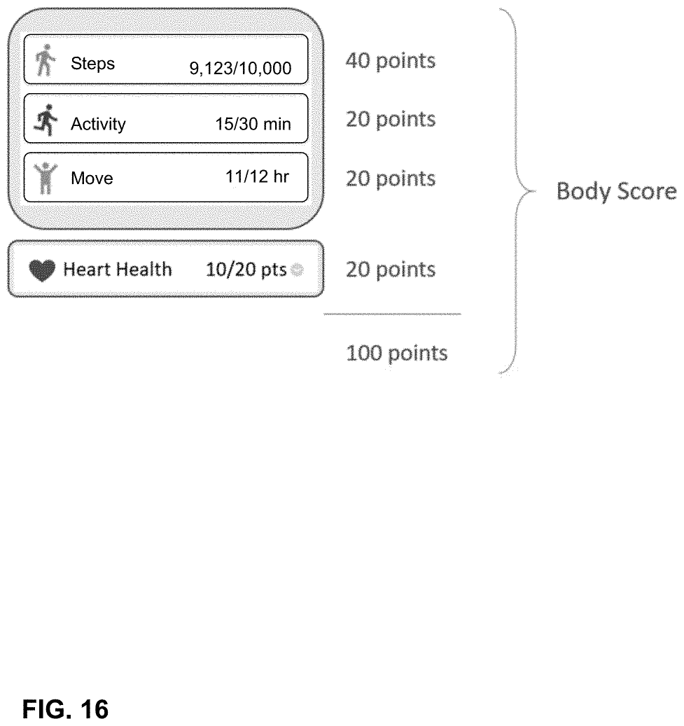

FIG. 16 is a conceptual diagram illustrating an example calculation technique for a body score, in accordance with one or more aspects of this disclosure. In the example of FIG. 16, a body score may have a step sub-component, an activity sub-component, and a move sub-component, as described elsewhere in this disclosure. Additionally, as shown in the example of FIG. 16, the body score may have a heart health sub-component that contributes points to a point total for the body score. In the example of FIG. 16, the steps sub-component has a maximum of 40 points, the activity sub-component has a maximum of 20 points, the move sub-component has a maximum of 20 points, and the heart health sub-component has a maximum of 20 points, for a total of 100 points. In some examples, the computing system may increase a point total for the heart health component for using features (e.g., heart rate determination, heart rate recovery) a given number of times per day. In some examples, to accommodate the additional heart health sub-component of the body score, circular diagram 910 (FIG. 9) may be changed to include a segment for the heart health sub-component, and there may be a feature in addition to features 904, 906, and 908 for the heart health sub-component.

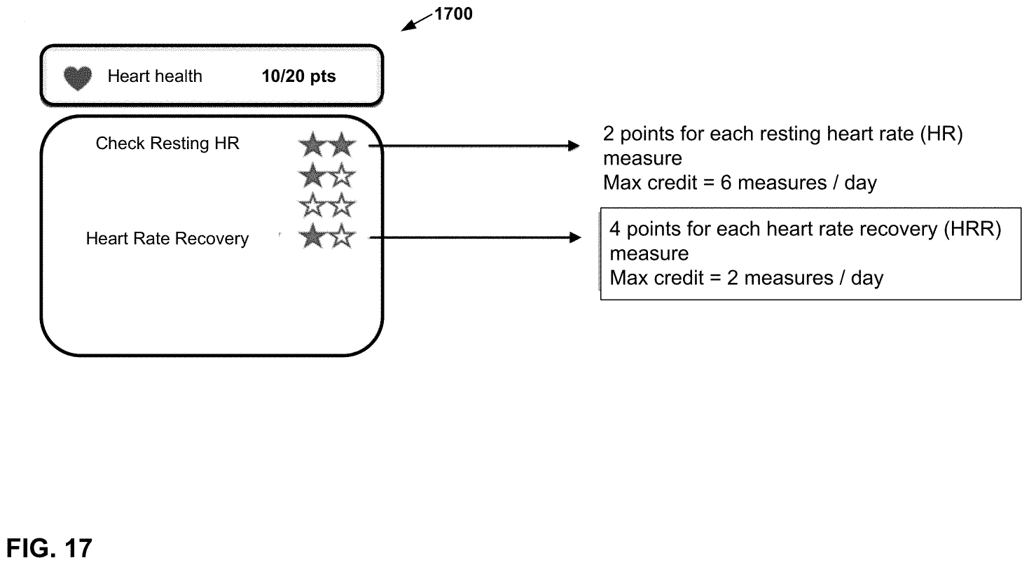

FIG. 17 is a conceptual diagram illustrating an example user interface feature 1700 for indicating values of components of heart health sub-component, in accordance with one or more aspects of this disclosure. In the example of FIG. 16, the heart health sub-component is based on the number of times the user of hearing instruments 102 checks their resting heart rate during a time period and the number of times the user performs the heart rate recovery routine during the time period. In the example of FIG. 17, the computing system awards the user a star (which may be worth x₁ points, where x₁ is 2 in the example of FIG. 17) for each time the user checks their resting heart rate in the time period (which in the example of FIG. 17 is one day), up to a maximum of x₂ stars (which in the example of FIG. 17 is six stars). Furthermore, in the example of FIG. 17, the computing system may award the user a star (which may be worth y₁ points, where y₁ is 4 in the example of FIG. 17) for each time the user checks their resting heart rate in the time period (which in the example of FIG. 17 is one day), up to a maximum of y₂ stars (which in the example of FIG. 17 is 2 stars).

In some examples, the resting heart rate is based on user-initiated measurements and also user-mediated classification (i.e., the user of hearing instruments 102 determines whether the user is at rest). For instance, in some examples, the user may be trusted to take measures of their heart rate while at rest. In some examples, the user may be rewarded for checking their heart rate at any time.

In some examples, the resting heart rate and/or heart rate recovery are measured automatically (e.g., on a periodic basis). In other words, the user may not need to initiate measurements of the user's heart rate and/or heart rate recovery. In some such examples, goal-based rewards may be used. However, in examples where the user initiates the measurements (e.g., of the user's heart rate) automatically, the user may be aware that such measurements may consume battery power, and therefore may disable the measurements in order to avoid the inconvenience of needing to recharge the batteries of hearing instruments 102 as frequently. Providing rewards, such as points, may help to incentivize the user to allow automatic measurements or manually perform measurements.

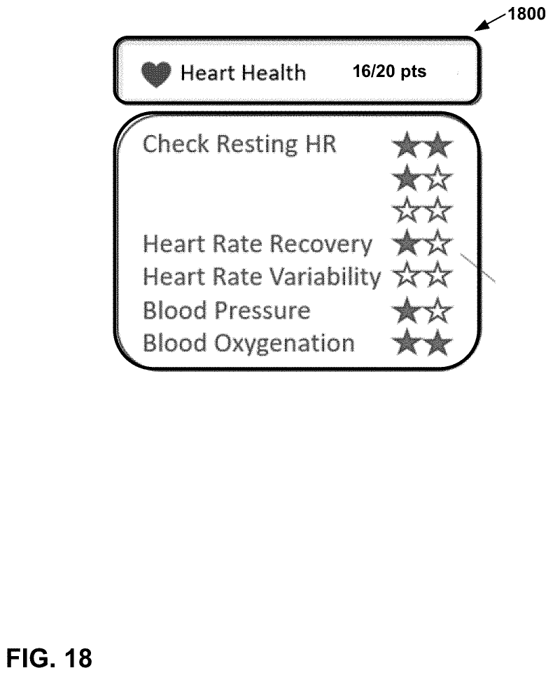

FIG. 18 is a conceptual diagram illustrating an example user interface feature 1800 for indicating values of components of heart health sub-component, in accordance with one or more aspects of this disclosure. In the example of FIG. 18, a scoring system is based on the number of times the user of hearing instruments 102 checks their resting heart rate, heart rate recovery, heart rate variability, blood pressure, and blood oxygenation. In other examples, in addition to or in place of one or more of these, the scoring system may be based on the number of times the user checks of the user's heart rate while the user's heart rate is elevated. In the example of FIG. 18 and other examples, the computing system may increase a point total (and/or number of stars) based on data such as current or historical measures. Such data may be housed (e.g., presented) in a separate cardiovascular section, and not in the body score section of the user interfaces.

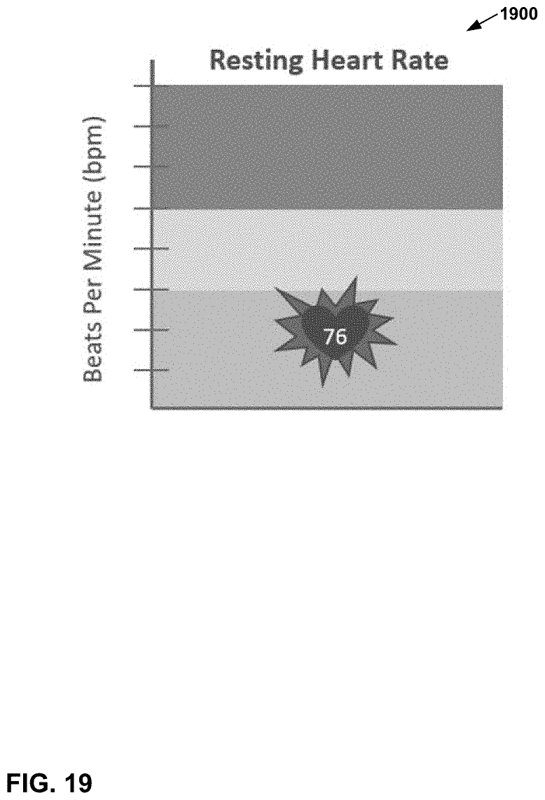

FIG. 19 is a conceptual diagram illustrating an example user interface feature 1900 for a goal-based heart health component in accordance with one or more aspects of this disclosure. In a scoring structure, such as that of FIG. 16, where heart health is a sub-component of the body score, the computing system may increase a point total in the heart health sub-component of meeting cardiovascular goals. The computing system may increase the point total of the heart health sub-component for achieving cardiovascular goals in addition to or as an alternative to increasing the point total of the heart health sub-component for taking measurements (e.g., heart rate measurements, heart rate recovery, etc.). Example cardiovascular goals may include one or more of: resting heart rate being within a particular range, exercise heart rate being above a given number of beats per minute, a heart rate elevated due to exercise N times per day (where N is a positive number), a heart rate recovery score in a particular range, a heart rate recovery score lower than a rolling average of heart rate recovery scores, a heart rate recovery score being lower by some percentage relative to earlier heart rate recovery scores, and so on.

In the example of FIG. 19, user interface feature 1900 shows a heart icon as being in different bands depending on the beats per minute of the user of hearing instruments 102.

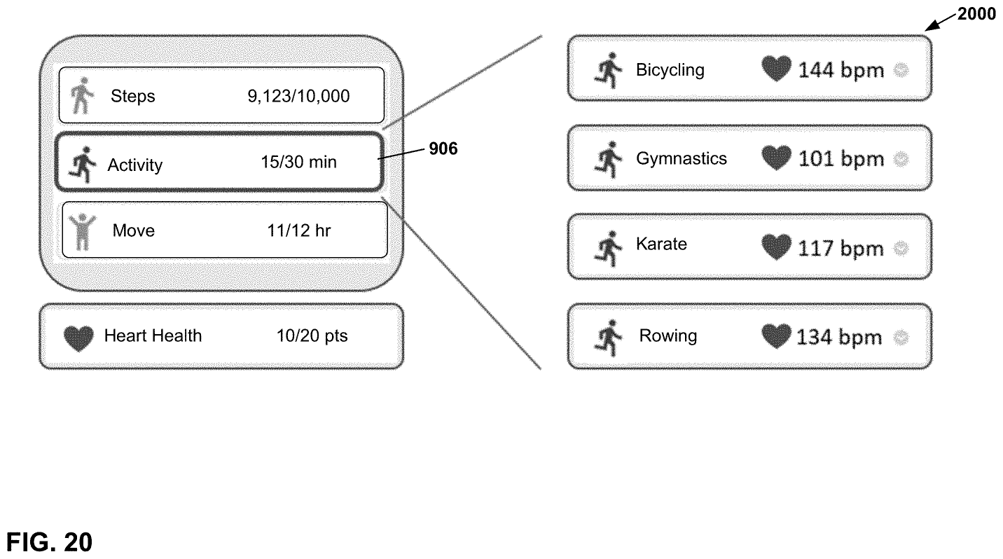

FIG. 20 is a conceptual diagram illustrating an example expanded activity sub-component 2000 in which heart rate measurements are indicated for different types of activities in accordance with one or more aspects of this disclosure. In the example of FIG. 20, heart rate measurements are indicated for bicycling, gymnastics, karate, and rowing activities. More, fewer, or different activities (e.g., running, weightlifting, cross-training, etc.) are possible. In some examples, the heart rates indicated for the activities may be an average or peak of heart rates measured while the user is performing the activities. In some examples, one or more of hearing instruments 102 may measure the heart rate of the user when a level of remaining power in a power source (e.g., power source 312) is above a threshold. In some examples, the computing system may have a separate heart health history for each activity.

In some examples, a computing system may output the expanded activity sub-component 2000 for display in response to receiving an indication of user selection of the feature 906 for the activity sub-component. The computing system may receive an indication of user input indicating which actions or activities the user is performing or was performing when measuring their heart rate.

In some examples, other types of data may contribute to one or more measures of the wellness of the user of hearing instruments 102. For example, the computing system may use signals from one or more of heart-related sensors 1124 (FIG. 11) of hearing instrument 1100 to generate arrhythmia-related information that may be used to assess a risk that the user has experienced one or more types of cardiac arrhythmia, such as atrial fibrillation. For instance, in this example, a set of electrodes may be built into one or more of hearing instruments 102. In some examples, the sets of electrodes are included in a behind-the-ear portion of hearing instruments 102 or another portion of hearing instruments 102.

In some examples, the computing system may use signals from the set of electrodes to generate an electrocardiogram (ECG) that tracks electrical activity of the user's heart. Furthermore, in some examples, the computing system may output the ECG for display (e.g., in a user interface of companion application 424). To analyze the signals from the set of electrodes, the computing system may apply an algorithm for detecting QRS complexes. Example algorithms for detecting QRS complexes include the Pan-Tompkins algorithm and algorithms based on the Hilbert transform. The computing system may analyze the QRS complexes to determine the presence or absence of P waves. The absence of P waves in combination with a rapid heart rate (e.g., greater than 100 beats per minute) may correspond to a high risk that the user has experienced an occurrence of atrial fibrillation. In some examples, the computing system may apply a machine learning system, such as a neural network, to the signals to determine risks that the user has experienced an occurrence of a cardiac arrhythmia.

In some examples, the computing system may output a message (e.g., in a user interface of companion application 424, as a text message, as an in-ear audio message, etc.) to the user in response to determining that the computing system has determined that there is an adequately high risk that the user has experienced or is experiencing a cardiac arrhythmia. For instance, in one example, the computing system may determine that there is an adequately high risk that the user has experienced or is experiencing atrial fibrillation if there is an absence of P waves and a rapid heart rate. In another example, the computing system may determine that there is an adequately high risk that the user has experienced or is experiencing periods of asystole if no heart rate can be detected. In some examples, the computing system may output such a message to a 3^{rd} party, such as a monitoring service, a physician, a caregiver, a family member, or another type of person.

In accordance with a technique of this disclosure, the computing system may use arrhythmia-related information as part of one or more measures of the health of the user of hearing instruments 102. For instance, the arrhythmia-related information may be integrated into a heart health score for the user and/or a body score for the user. Thus, in some examples, the computing system may determine the user's heart health score and/or body score based at least on part on arrhythmia-related information. For instance, in some examples, the computing system may reduce the user's heart health sub-component if the computing system determines that a sufficiently high risk that the electrical signals represent an occurrence of a cardiac arrhythmia.

In some examples, the user may initiate an arrhythmia analysis process that measures electrical signals of the user's heart and analyzes the electrical signals to determine a risk that the electrical signals represent an occurrence of a cardiac arrhythmia. For instance, similar to feature 1306 in the example of FIG. 13, a user interface may include a feature for initiating the arrhythmia analysis process. Thus, in response to receiving an indication of user input to select this feature, the computing system may initiate the arrhythmia analysis process. Performing the arrhythmia analysis process in response to user input may help hearing instruments 102 conserve energy relative to systems in which hearing instruments 102 automatically or continuously perform the arrhythmia analysis. However, placing responsibility on the user to initiate the arrhythmia analysis process may result in the user forgetting to regularly initiate the arrythmia analysis process. Accordingly, the computing system may use score-based techniques to incentivize the user to initiate the arrhythmia analysis process.

Similar to the example of FIG. 15, the computing system may add bonus points to the body score of the user when the user initiates the arrhythmia analysis process. In an example similar to that of FIG. 16, the user's body score has a heart health sub-component. The computing system may assign points contributing to the heart health sub-component based on the user initiating the arrhythmia analysis process. In examples similar to that of FIG. 17 and FIG. 18, the computing system may increase a number of awarded stars in response to the user initiating the arrhythmia analysis process. In some examples, the computing system may use a heart health score separate from the body score.

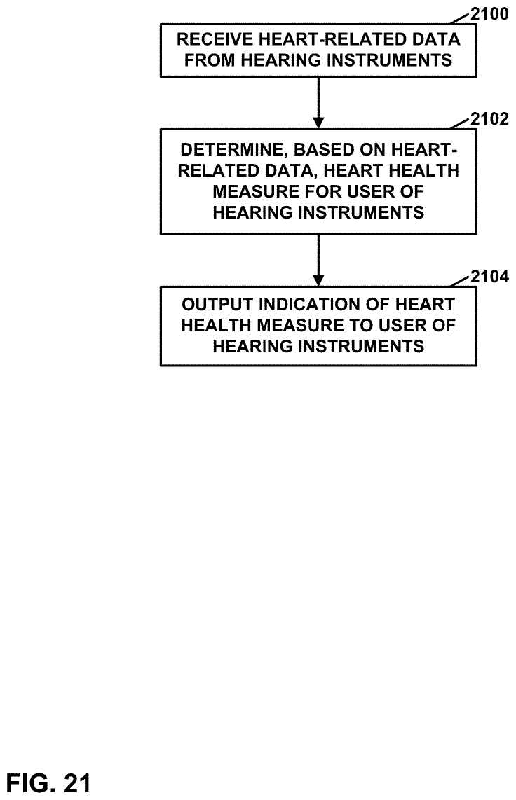

FIG. 21 is a flowchart illustrating an example operation, in accordance with one or more aspects of this disclosure. In the example of FIG. 21, a computing system may receive heart-related data from one or more hearing instruments (2100). The computing system comprises a set of one or more electronic computing devices. For instance, the computing system may include one or more computing devices of computing system 104 (FIG. 1), hearing instruments 102, and/or one or more other devices. As described elsewhere in this disclosure, the heart-related data may include a signal from a PPG sensor of the one or more hearing instruments, a signal from an IMU of the one or more hearing instruments, one or more signals from ECG electrodes of the one or more hearing instruments, and/or other signals.

Furthermore, in the example of FIG. 21, the computing system may determine, based on the heart-related data received from the one or more hearing instruments, a heart health measure for a user of the one or more hearing instruments (2102). The heart health measure is an indication of one or more aspects of a health of a heart of the user.

The computing system may determine the heart health measure in any of various ways. For instance, in one example, the computing system may determine a plurality of sub-components of the heart health measure. For instance, the sub-components may include one or more of a heart rate sub-component and a heart rate recovery sub-component. The computing system may determine the heart rate sub-component as a total number of times the user initiated the process to check the user's heart rate. The computing system may determine the heart rate recovery sub-component as a total number of times the user initiated the process to check the user's heart rate recovery. In this example, the computing system may determine the heart health measure based on the plurality of sub-components of the heart health measure. For instance, the computing system may determine the heart health measure as a total of points for the sub-components.

The computing system may output an indication of the heart health measure to the user of the hearing instruments (2104). For instance, the computing system may output the indication of the heart health measure in a GUI as described elsewhere in this disclosure. In some examples, the computing system may send a message (e.g., to the user of the hearing instruments or a 3^{rd} party) indicating the heart health measure.

In some examples, the computing system may determine, based on the data received from the one or more hearing instruments, a body measure for the user. The body measure may be an indication of physical health of the user. The computing system may output an indication of the body measure. In some examples, the computing system determines the body measure in accordance with any of the examples provided elsewhere in this disclosure, or others. Thus, in some examples, the computing system may output indications of both the heart health measure and a separate the body measure. In other examples, the heart health measure is a sub-component of the body measure. For instance, in such examples, the computing system may add together points for each of the sub-components of the body measure to determine the body measure. Furthermore, in some examples where the body measure and the heart health measure are separate, the computing system may determine a wellness measure (e.g., a Thrive Score) based on the body measure and the heart health measure. In such examples, the wellness measure may be an indication of an overall wellness of the user. For instance, the computing system may add together the body measure and the heart health measure.

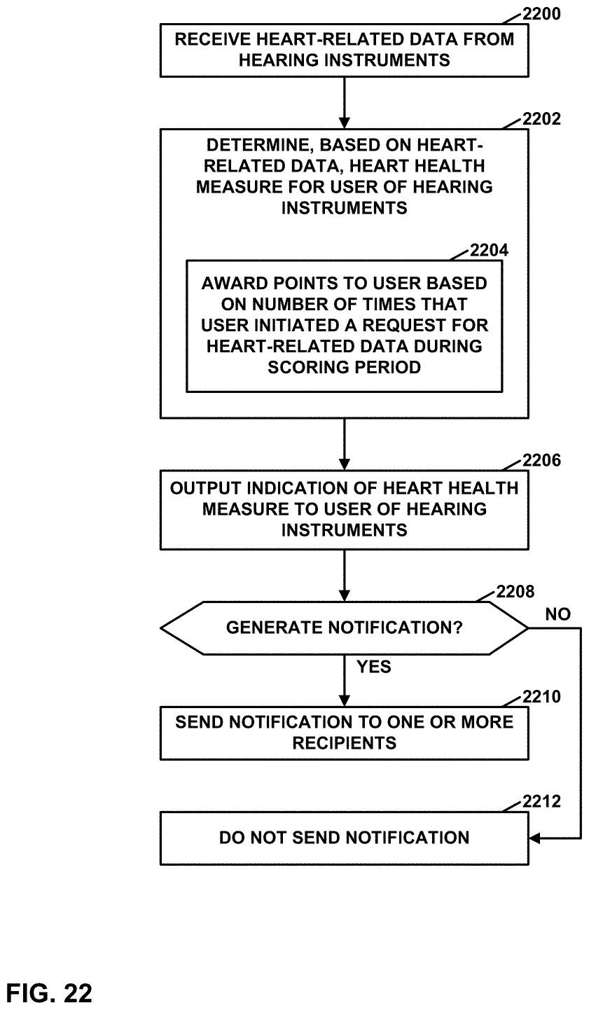

FIG. 22 is a flowchart illustrating an example operation, in accordance with one or more aspects of this disclosure. The example of FIG. 22 may be considered an extension of the operation of FIG. 21. In the example of FIG. 22, a particular hearing instrument in the set of hearing instruments is configured to receive a request for the heart-related data and wirelessly transmit the heart-related data in response to the request. The request may be initiated by the user of the one or more hearing instruments. Furthermore, in the example of FIG. 22, the particular hearing instrument uses electrical energy from a battery (e.g., power source 1114) internal to the particular hearing instrument to wirelessly transmit the heart-related data to the computing system in response to the request.

In the example of FIG. 22, the computing system receives the heart-related data from one or more hearing instruments, including the particular hearing instrument (2200). Furthermore, the computing system may determine, based on the heart-related data, a heart health measure for the user of the one or more hearing instruments (2202). In the example of FIG. 22, as part of determining the heart health measure, the computing system may increase a point total of the user by one or more points based on a number of times that the user initiated a request for the heart-related data during a scoring time period (2204). For instance, the computing system may increase the point total by 2 points for each time the user initiated a process to check the user's resting heart rate, up to a first limit; and may increase the point total by 2 points for each time the user initiated a process to check the user's heart rate recovery. The computing system may output an indication of the heart health measure to the user of the hearing instruments (2206). The computing system may output the indication of the heart health measure in accordance with any of the examples provided elsewhere in this disclosure, and others.

Furthermore, in the example of FIG. 22, the computing system may determine, based on the heart-related data, whether to generate a notification (2208). The notification may be with regard to an aspect of a cardiac health of the user of the hearing instruments. For instance, the computing system may make a determination to generate the notification in response to determining that the user's resting heart rate or heart rate recovery is outside a healthy range, in response to determining that the user has experienced a heart arrhythmia, or in response to other conditions. In response to making the determination not to generate the notification (“NO” branch of 2212), the computing system does not send the notification (2212).

However, in response to making the determination to generate the notification (“YES” branch of 2208), the computing system may send the notification to one or more recipients (2210). For instance, the computing system may send the notification to the user of the hearing instruments or a third party. The third party may be a party other than the user of the hearing instruments and other than a provider of the computing system.

In some examples, the computing system may use data from one or more sensors of hearing instruments 102 or other devices to generate stress-related data. For instance, the computing system may use data from stress-related sensors 1126 of hearing instrument 1100 (FIG. 11). The computing system may use the stress-related data to determine an emotional stress level of the user. Furthermore, in some examples, the computing system may use the determined emotional stress level to encourage the user to perform activities to manage their levels of emotional stress.

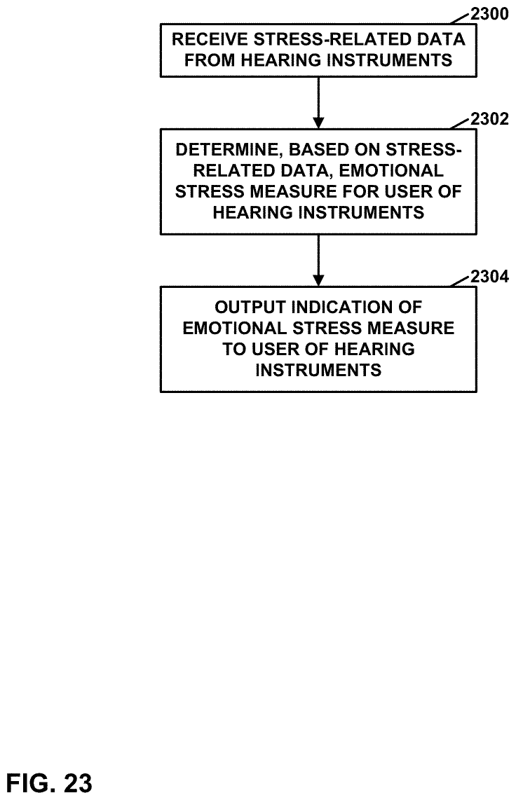

For instance, FIG. 23 is a flowchart of an example operation in which the computing system uses stress-related data, in accordance with one or more aspects of this disclosure. In the example of FIG. 23, the computing system may receive stress-related data from one or more of hearing instruments 102 (2300). For instance, the computing system may receive the stress-related data via a wireless transmission from one or more of hearing instruments 102. In some examples, such as examples where the computing system is implemented as one or more processors in a hearing instrument, the processors may receive the stress-related data from one or more sensors of the hearing instrument. The stress-related data may include one or more of data regarding meditation practices of the user of the hearing instruments, data regarding physical activity levels of the user of the hearing instruments, or data regarding a respiration rate of the user of the hearing instruments, or other types of data relating to a stress level of the user of hearing instruments 102.

Additionally, in the example of FIG. 23, the computing system may determine, based on the stress-related data, an emotional stress measure of a user of the one or more hearing instruments (2302). The emotional stress measure being an indication of one or more aspects of a level of emotional stress of the user. Additionally, the computing system may output an indication of the emotional stress measure to the user of the hearing instruments (2304).

The computing system may determine the emotional stress measure in one of various ways. For instance, in some examples, a set of sensors in hearing instruments 102 may generate signals that the computing system may use to detect a respiration rate of the user. Higher respiration rates, especially when not associated with physical movement, are often a sign of emotional stress. In other words, when people are emotionally stressed, they typically breathe more but this higher respiration rate is not associated with exercise or other physical activity. IMUs, microphones, and other types of sensors may generate the signals that the computing system may use to detect the respiration rate of the user. In some examples, the computing system may detect the respiration rate of the user by based on a signal from an inward-facing microphone on the medial end of a receiver in the ear canal of the user. In such examples, an acoustic signature of respiration may be defined through supervised machine learning, and the computing system may apply algorithms that classify internal body noise as inhalation or exhalation. The computing system may use patterns of inhalation and exhalation over time to measure respiration rate. In some examples, the computing system may deduct points from an emotional stress measure for each instance where the computing system determines that the user has experienced an episode of emotional stress.

The computing system may use signals from other types of sensors in hearing instruments 102 to determine an emotional stress level of the user of hearing instruments 102. For example, the sensors in hearing instruments 102 may include electrodes configured to generate EEG signals. Certain patterns of EEG signals are associated with relaxation and stress. For instance, EEG signals that exhibit wave patterns in the alpha band are associate with relaxation. In contrast, EEG signals that exhibit wave patterns in the beta band are associated with anxious thinking and active concentration. In adults, EEG signals that exhibit wave patterns in the theta band may be associated with meditation.

In some examples, the stress-related information (i.e., stress-related data) may include self-reported information about the user's stress management practices. For example, the computing system may receive indications of user input indicating amounts of time that the user spent practicing meditation. In some examples, the computing system may receive information about the user's stress management practices from one or more software applications or devices. For example, a software application for meditation may provide information to the computing system indicating amounts of time that the user spent practicing meditation. In some examples, the software application for meditation provides audio data through hearing instruments 102.

In some examples, the computing system may use the stress-related information to determine a stress level of the user of hearing instruments. For example, the computing system may apply a neural network to the stress-related information to generate information indicative of the emotional stress level. In this example, the neural network may output a score indicative of the user's stress level.

The computing system may use stress-related information as part of one or more measures of the health of the user of hearing instruments 102. For instance, the computing system may add bonus points to a brain score of the user if the user is taking steps to manage stress. For example, the computing system may add a given number of bonus points to the brain score of the user if the stress-related information indicates that the user performs at least a particular number of minutes of meditation. In some examples, the computing system may add a particular number of bonus points to the brain score of the user if the stress-related information indicates that the user's heart rate did not rise above a particular limit while not performing a physical activity.

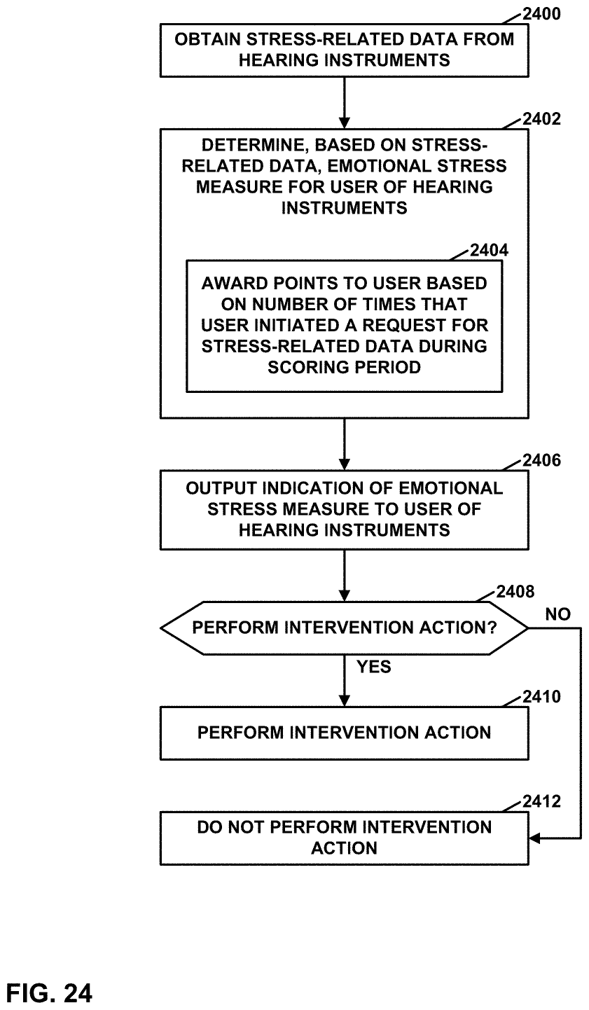

FIG. 24 is a flowchart of an example operation in which the computing system uses stress-related data to determine whether to perform an intervention action, in accordance with one or more aspects of this disclosure. The example of FIG. 24 may be considered an extension of the operation of FIG. 23. In the example of FIG. 24, the computing system may obtain stress-related data from one or more hearing instruments (2400).

In the computing system may obtain the stress-related data in one or more of various ways. For instance, in one example, a particular hearing instrument in a set of hearing instruments (e.g., hearing instruments 102) may be configured to receive a request for the stress-related data and wirelessly transmit the stress-related data in response to the request. In this example, the request may be initiated by the user of the one or more hearing instruments. Furthermore, in this example, the particular hearing instrument uses electrical energy from a battery internal to the particular hearing instrument to wirelessly transmit the stress-related data to the computing system in response to the request. In some examples, the computing system may obtain the physiological data via communication channels (e.g., communication channels 1116), via a wireless communication link, or in another manner.

Additionally, the computing system may determine, based on the stress-related data, an emotional stress measure of a user of the one or more hearing instruments (2402). As before, the emotional stress measure is an indication of one or more aspects of a level of emotional stress of the user.

In the example of FIG. 24, as part of determining the emotional stress measure of the user, the computing system may increase a point total of the user by one or more points based on a number of times that the user initiated a request for the stress-related data during a scoring time period (2404). For example, the computing system may increase the point total by a point (which may be represented by a star or other icon) for each time the user initiates a request for the stress-related data during the scoring period, up to a given maximum number of points. In another example, the computing system may increase the point total by a given number of points (e.g., 5 points) for each time the user initiates a request for the stress-related data during the scoring period.

In some examples, the computing system may use the point total of the user for initiating requests for stress-related data to determine a cognitive wellness score for the user. For instance, the computing system may add the points awarded for initiating requests for stress-related data to the points awarded as described elsewhere in this disclosure, to determine the cognitive wellness score.

In some examples, the computing system may determine, based on the stress-related data, whether the user of the hearing instruments has achieved one or more stress management goals for the user of the hearing instruments. For example, the stress-related data may indicate an amount of time the user used hearing instruments 102 to perform guided meditation. In this example, the computing system may determine that the user has achieved a stress management goal if the user has used hearing instruments 102 to perform at least a particular number of minutes of guided meditation in a particular time period. In another example, a stress management goal may be to maintain a stable heart rate while at rest or resting respiration rate over a period of time. In this example, the IMU and associated activity-classification algorithms may be used to determine when the user is at rest. The computing system may use one or more sensors of the hearing instruments to determine values of the physiological measures of interest, such as respiration rate. Furthermore, in one example, one or more of the hearing instruments may include a sensor for detecting the galvanic skin response, which is a measure of skin conductance and may be used to measure physiological arousal, which is a proxy for stress. In this example, a stress management goal may be to keep the amount of time during which the user experiences physiological arousal to below certain thresholds. The computing system may increase the point total by one or more points to the user of the hearing instruments based on the user of the hearing instruments achieving the one or more stress management goals.

Furthermore, in the example of FIG. 24, the computing system may output an indication of the emotional stress measure to the user of the hearing instruments (2406). The computing system may output the indication of the emotional stress measure in accordance with any of the examples provided with respect to action 2304 of FIG. 23, and others.

In the example of FIG. 24, the computing system may determine, based on the stress-related information, whether to perform an intervention action (2408). An intervention action encourages the user of hearing instruments 102 to take action to manage the user's stress level. For example, in response to determining that the user's stress level is above a particular threshold, the computing system may cause hearing instruments 102 to output audio signals that ask the user whether they would like to perform a stress-management action. For example, the computing system may cause hearing instruments 102 to output audio signals with a regular rhythm, like the sound of a metronome, with which the user of hearing instruments 102 can synchronize their breathing. In other words, the computing system may instruct hearing instruments 102 to output a metronomic rhythm and an audio message encouraging the user of the hearing instruments to synchronize their breathing to the metronomic rhythm. Intentional slowing of the respiration rate may decrease the user's heart rate and may reduce the user's feelings of stress. In some examples, the computing system may cause hearing instruments 102 to send a message to the user of hearing instruments 102 suggesting stress management techniques. For instance, the computing system may cause hearing instruments 102 to output audio affirmations and encouragement to the user of hearing instruments 102. In some examples, the computing system may cause hearing instruments 102 to output audio of encouragement to practice meditation.

In some examples, as part of performing a notification action, the computing system may send one or more messages to an account associated with the user of hearing instruments 102, where the messages provide information about techniques to reduce stress. For example, the computing system may send email messages to an email account of the user, SMS messages to a phone number of the user, and so on. In some examples, the computing system may cause a GUI (e.g., a GUI of companion application 424 (FIG. 4)) to contain messages about techniques for reducing stress.

In some examples, as part of performing a notification action, the computing system may send a notification to a third party. The third party may be a party other than the user of hearing instruments 102 and other than a provider of the computing system. In response to receiving such a notification, the third party may advise the user of hearing instruments 102 on ways to reduce the user's stress levels.

The computing system may make the determination of whether to perform the intervention action in any of one or more ways. For example, the computing system may implement a machine learning model (e.g., a neural network) that takes the stress-related information as input and outputs an indication of whether to perform the intervention action. In some examples, the computing system may apply a rules engine that evaluates a set of rules. In such examples, a rule may indicate that an intervention action is to be performed if the stress-related information indicates that the user experiences more than a given number of stressful episodes during a particular time period (e.g., day, week, hour, etc.). In some examples, a rule may indicate not to perform an intervention action if the stress-related data indicates that the user is performing actions to reduce the user's stress level. For instance, the rule may indicate not to perform an intervention action if the stress-related data indicates that the user is exercising meditation, performing regular physical exercise, or performing other activities that are associated with stress reduction.

In response to making a determination to perform the intervention action (“YES” branch of 2408), the computing system may perform the intervention action (2410). For instance, the computing system may send an email message, output an audio message, or perform any of the other example intervention actions described elsewhere in this disclosure, or others. Otherwise, in response to making a determination not to perform the intervention action (“NO” branch of 2408), the computing system does not perform the intervention action (2412).

In some examples, in addition to or as an alternative to using heart-related information (i.e., heart-related data) to determine one or more measures of the physical or mental wellness of the user of hearing instruments 102, the computing system may use the cardiovascular-information for purposes of fall detection. Falls are a common cause of serious injury, especially among the elderly. People with certain cardiovascular conditions are at a greater risk of falling. For instance, a user is more likely to fall if there is too little oxygenated blood reaching the user's brain. The user may have too little oxygenated blood reaching the user's brain for a variety of reasons, such as when the user has low blood pressure, is experiencing a cardiac arrhythmia, has a heart rate that is abnormally fast or slow, or has an abnormally fast or slow respiration rate.

The computing system may use one or more of various techniques for detecting a fall. For instance, in some examples, the computing system may determine whether the user has fallen based on signals from IMU 326. In some examples, the computing system uses information from one or more photoplethysmography (PPG) sensors of hearing instruments 102 to detect whether the user has fallen (e.g., as described in U.S. patent application Ser. No. 16/230,110, filed Dec. 21, 2018). One challenge associated with fall detection is minimizing false alarms while reducing the chances of a real fall not being detected. False alarms can be inconvenient and waste the resources of first responders. However, the user may not receive needed help if the fall detection algorithm did not detect a real fall.

As shown in the example of FIG. 11, hearing instrument 1100 may include a fall detection system 2028 that is configured to detect whether the user of hearing instrument 1100 has fallen. In other examples, fall detection system 2028 may be implemented in hardware or in a device separate from hearing instrument 1100.

In accordance with a technique of this disclosure, the computing system may use the heart-related information to determine whether the user of hearing instruments 102 is at an increased risk of falling. In response to determining that the user is at an increased risk of falling, the computing system may change a sensitivity level of the fall detection algorithm. For instance, if the computing system determines that the user's blood pressure is low, that the user is experiencing a cardiac arrhythmia, or that the user's heart rate is abnormally high or low, the computing system may increase the sensitivity level of the fall detection algorithm. In general, a higher sensitivity level reduces the likelihood that the fall detection algorithm does not detect a fall, but also increases the likelihood of false detections.

In some examples, the fall detection algorithm may generate a value indicating a likelihood that the user has fallen. For instance, in one example, a neural network may be trained to generate the value. In this example, the neural network may take various types of data as input. For instance, the neural network may take IMU data, PPG data, or other types of data as input. Furthermore, in this example, the computing system may determine that the user has fallen if the value is greater than a threshold. Changing the sensitivity level of the fall detection algorithm based on the cardiovascular information may comprise adjusting the threshold.

In some examples, the computing system may use other information in addition to or as an alternative to the cardiovascular information to determine whether the user of hearing instruments 102 is at an increased risk of falling. For example, one or more EEG electrodes may be integrated into hearing instruments 102. In this example, the computing system may monitor EEG signals from the EEG electrodes for signs that the user might be experiencing or is at risk of experiencing an epileptic seizure. For instance, the computing system may monitor the EEG signals for spikes or sharp waves that may represent seizure activity or interictal activity. Accordingly, the computing system may increase the sensitivity level of the fall detection algorithm in response to determining that the user might be experiencing or is at risk of experiencing an epileptic seizure.

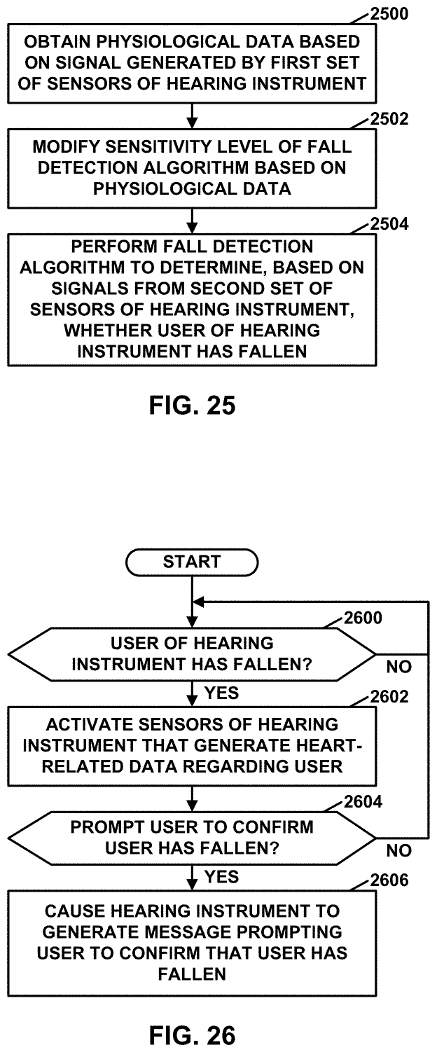

FIG. 25 is a flowchart illustrating an example operation, in accordance with one or more aspects of this disclosure. In the example of FIG. 25, a computing system may obtain physiological data based on signals generated by a first set of one or more sensors of a hearing instrument (2500). The computing system may include computing system 104, hearing instruments 102, and/or other devices or systems. For instance, in some examples, the computing system is included in a hearing instrument.

The computing system may obtain the physiological data via communication channels (e.g., communication channels 1116), via a wireless communication link, or in another manner. The physiological data includes heart-related data. For instance, the heart-related data may include one or more of data indicating a blood pressure of the user of the hearing instrument, data indicating a heart rate of the user of the hearing instrument, ECG data for the user of the hearing instrument, data regarding a potential cardiac arrythmia of the user of the hearing instrument, or other types of data related to the heart of the user. In some examples, the physiological data may include data that is not directly heart related. For instance, the physiological data may include one or more of EEG data generated by one or more electrodes included in the hearing instrument, data indicating a respiration rate of the user of the hearing instrument, data indicating a level of physical activity of the user of the hearing instrument, or other types of data regarding the user.

Furthermore, in the example of FIG. 25, the computing system may modify a sensitivity level of a fall detection algorithm based on the physiological data (2502). For example, the computing system may implement a machine learning model (e.g., a neural network) that is trained to accept the physiological data as input and to generate data indicating a sensitivity level. In this example, the computing system may modify the sensitivity level of the fall detection algorithm to correspond to the sensitivity level indicated by the output of the machine learning model. In this example, the machine learning model may be trained using training data that map sets of physiological data to sensitivity levels. In some examples, the computing system may implement a rules engine that evaluates rules for mapping values of physiological parameters to sensitivity levels. The sensitivity determination algorithm may use inputs from various physiological sensors to assess whether the user is at increased risk of falling relative to an average, stable user. For example, those inputs may include ambulation rate, IMU-based measures of balance, activity levels, EEG-based measures of epileptic seizure, etc. If a user is deemed to be at higher risk, the computing system may increase the sensitivity, thereby increasing the likelihood that the computing system detects a fall would be detected when the occurs, and that an alert for assistance would be sent.

The computing system may perform the fall detection algorithm to determine, based on signals from a second set of one or more sensors of the hearing instrument, whether a user of the hearing instrument has fallen (2504). For instance, in one example, the computing system may generate a confidence value that indicates a level of confidence that the user of the hearing instrument has fallen. In this example, the computing system may determine that the user has fallen based on the confidence value being greater than the sensitivity level. In this example, the computing system may generate the confidence level in various ways. For instance, in an example where the second set of sensors includes a PPG sensor, the computing system may determine DC component values of a PPG signal and determine differences between the DC component values. An abrupt decrease in the DC component values may correspond to the user falling. In this example, the computing system may determine the confidence value based on a mapping of differences to allowable levels of confidence. For instance, greater differences may be mapped to greater levels of confidence. In another example, a neural network may be trained to generate the level of confidence.

In some examples, the computing system modifies the sensitivity level of the fall detection algorithm in response to an indication of user input. For example, hearing instruments 102 may detect the sound of the user's voice and the computing system may detect the user saying something to the effect that they feel unsteady. In this example, the computing system may use a voice recognition toolkit to analyze the user's voice for words or phrases that indicate that the user feels unsteady. In response to detecting words or phrases that indicate that the user feels unsteady, the computing system may increase the sensitivity level of the fall detection algorithm. In some examples, the computing system may decrease the sensitivity level of the fall detecting algorithm in response to an indication of user input. For instance, if the user is planning to participate in a certain type of activity (e.g., judo), the user may provide an indication of user input to decrease the sensitivity level of the fall detection algorithm.

In some examples, the computing system may use cardiovascular information regarding the user of hearing instruments 102 to determine whether to ask the user whether assistance is needed after determining that the user has fallen. For example, the computing system may use a fall detection algorithm to determine that the user has fallen. In this example, the computing system may cause one or more of hearing instruments 102 to output an audio question that asks the user to indicate whether the user needs assistance. The user may respond in one or more of various ways, such as by providing a spoken response, performing a head gesture, or providing another type of response. In some examples, the computing system may automatically request assistance if the user does not respond within a particular amount of time.

FIG. 26 is a flowchart illustrating an example operation, in accordance with one or more aspects of this disclosure. In the example of FIG. 26, the computing system may determine whether a user of a hearing instrument has fallen (2600). For example, the computing system may analyze data from IMU 1118 to identify a pattern of movement consistent with a fall. For instance, data from IMU 1118 may indicate a sudden downward acceleration and abrupt deceleration. In response to determining that the user has not fallen (“NO” branch of 2600), the computing system may continue a process of determining whether the user has fallen.

In response to determining that the user has fallen (“YES” branch of 2600), the computing system may activate one or more sensors of hearing instrument 1100 that generate heart-related data regarding the user (2602). For instance, the one or more sensors include a PPG sensor, ECG electrodes, or other types of sensors that generate heart-related data regarding the user. Activating the one or more sensors in response to determining that the user has fallen, instead of keeping the one or more sensors constantly active, may help to conserve electrical energy from power source 1114 of hearing instrument 1100. Conserving electrical energy may be important in hearing instruments because the space in hearing instruments for larger power sources is typically very constrained.

Subsequently, the computing system may determine, based on the heart-related data, whether to prompt the user to confirm that the user has fallen (2604). The computing system may determine whether to prompt the user in one or more of various ways.

For instance, in one example where the sensors include a PPG sensor, the computing system may determine, based on heart-related data generated by the PPG sensor, a heart rate of the user. For instance, the computing system may determine the heart rate of the user based on times between peaks of maximum blood perfusion. Additionally, the computing system may determine, based on the heart rate of the user being above a first threshold or below a second threshold, whether to prompt the user the confirm that the user has fallen. That is, the user is likely to have an elevated heart rate immediately after the user experiences a fall. Accordingly, the likelihood that the user has actually fallen may be higher if the computing system determines that the user has an elevated heart rate during a period that immediately follows a time at which the user is determined to have fallen. Moreover, a low heart rate may lead to fainting, which is a common cause of falls. Accordingly, the likelihood that the user has actually fallen may be higher if the computing system determine that the user has a depressed heart rate during a period that immediately follows a time at which the user is determined to have fallen.

In some examples where the sensors include a PPG sensor, the computing system may determine, based on heart-related data generated by the PPG sensor, a level of blood perfusion of the user. For instance, the computing system may determine the level blood perfusion that is mapped to a DC signal of a signal from the PPG sensor. Additionally, the computing system may determine, based on the level of blood perfusion of the user, whether to prompt the user to confirm that the user has fallen. For instance, the user's blood perfusion may decrease to a given level if the user has fallen from a standing position. Accordingly, if the computing system determines that the user has fallen, and also determines that the user's blood perfusion has decreased, the computing system hay prompt the user to confirm that the user has fallen.

In response to determining not to prompt the user to confirm that the user has fallen (“NO” branch of 2604), the computing system may continue the process of determining whether the user has fallen. In some examples, the computing system may perform one or more additional actions, such as requesting assistance, despite making the determination not to prompt the user to confirm that the user has fallen.

On the other hand, in response to making a determination to prompt the user to confirm that the user has fallen (“YES” branch of 2604), the computing system may cause the hearing instrument to generate a message prompting the user to confirm that the user has fallen (2606). For example, the computing system may cause the hearing instrument to output an audio message asking whether the user has fallen. In some examples, the computing system may output a haptic signal that prompts the user to confirm that the user has fallen.

The following is a non-limiting list of examples that are in accordance with one or more techniques of this disclosure.

### EXAMPLE 1A

A computer-implemented method comprising: receiving, by a computing system comprising a set of one or more electronic computing devices, heart-related data from one or more hearing instruments; determining, by the computing system, based on the heart-related data received from the one or more hearing instruments, a heart health measure for a user of the one or more hearing instruments, the heart health measure being an indication of one or more aspects of a health of a heart of the user; and outputting, by the computing system, an indication of the heart health measure to the user of the hearing instruments.

### EXAMPLE 2A

The computer-implemented method of example 1A, wherein: a particular hearing instrument in the set of hearing instruments is configured to receive a request for the heart-related data and wirelessly transmit the heart-related data in response to the request, wherein the request is initiated by the user of the one or more hearing instruments, the particular hearing instrument uses electrical energy from a battery internal to the particular hearing instrument to wirelessly transmit the heart-related data to the computing system in response to the request, determining the heart health measure comprises increasing, by the computing system, a point total of the user by one or more points based on a number of times that the user initiated a request for the heart-related data during a scoring time period; and the method further comprises: determining, by the computing system, based on the heart-related data, whether to generate a notification; and based on a determination to generate the notification, sending, by the computing device, the notification to one or more recipients.

### EXAMPLE 3A

The computer-implemented method of example 2A, wherein the one or more recipients include at least one of: the user of the hearing instruments, or a third party, wherein the third party is a party other than the user of the hearing instruments and other than a provider of the computing system.

### EXAMPLE 4A

The computer-implemented method of any of examples 1A-3A, wherein determining the heart health measure comprises: determining, by the computing system, a plurality of sub-components of the heart health measure; and determining, by the computing system, the heart health measure based on the plurality of sub-components of the heart health measure.

### EXAMPLE 5A

The computer-implemented method of example 4A, wherein determining the plurality of sub-components comprises one or more of: determining, by the computing system, a heart rate sub-component, or determining, by the computing system, a heart rate recovery sub-component.

### EXAMPLE 6A

The computer-implemented method of any of examples 1A-5A, further comprising: determining, by the computing system, based on the data received from the one or more hearing instruments, a body measure for the user, the body measure being an indication of physical health of the user; and outputting, by the computing system, an indication of the body measure.

### EXAMPLE 7A

The computer-implemented method of example 6A, wherein the heart health measure is a sub-component of the body measure.

### EXAMPLE 8A

The computer-implemented method of example 6A, further comprising determining a wellness measure based on the body measure and the heart health measure, the wellness measure being an indication of an overall wellness of the user.

### EXAMPLE 9A

The computer-implemented method of any of examples 1A-8A, wherein: the heart-related data from the one or more hearing instruments is based on one or more of: a signal from a photoplethysmography (PPG) sensor of the one or more hearing instruments, a signal from an inertial measurement unit (IMU) of the one or more hearing instruments, or one or more signals from electrocardiogram (ECG) electrodes of the one or more hearing instruments.

### EXAMPLE 1B

A computer-implemented method comprising: receiving, by a computing system comprising one or more electronic computing devices, stress-related data from one or more hearing instruments; determining, by the computing system, based on the stress-related data, an emotional stress measure of a user of the one or more hearing instruments, the emotional stress measure being an indication of one or more aspects of a level of emotional stress of the user; and outputting, by the computing system, an indication of the emotional stress measure to the user of the hearing instruments.

### EXAMPLE 2B

The computer-implemented method of example 1B, further comprising: a particular hearing instrument in the set of hearing instruments is configured to receive a request for the stress-related data and wirelessly transmit the stress-related data in response to the request, wherein the request is initiated by the user of the one or more hearing instruments, the particular hearing instrument uses electrical energy from a battery internal to the particular hearing instrument to wirelessly transmit the stress-related data to the computing system in response to the request, determining the emotional stress measure comprises increasing, by the computing system, a point total of the user by one or more points based on a number of times that the user initiated a request for the stress-related data during a scoring time period; the method further comprises: determining, by the computing system, based on the stress-related data, whether to perform an intervention action; and based on a determination to perform the intervention action, performing, by the computing device, the intervention action.

### EXAMPLE 3B

The computer-implemented method of example 2B, wherein performing the intervention action comprises sending a notification to a third party, wherein the third party is a party other than the user of the hearing instruments and other than a provider of the computing system.

### EXAMPLE 4B

The computer-implemented method of any of examples 2B-3B, wherein performing the intervention action comprises one or more of: instructing the one or more hearing instruments to output a metronomic rhythm and an audio message encouraging the user of the hearing instruments to synchronize their breathing to the metronomic rhythm, sending a message to the user of the hearing instruments suggesting stress management techniques.

### EXAMPLE 5B

The computer-implemented method of any of examples 2B-4B, wherein determining the emotional stress measure comprises: determining, by the computing system, based on the stress-related data, whether the user of the hearing instruments has achieved one or more stress management goals for the user of the hearing instruments; and increasing, by the computing system, the point total of the user by one or more points based on the user of the hearing instruments achieving the one or more stress management goals.

### EXAMPLE 6B

The computer-implemented method of any of examples 1B-5B, wherein the stress-related data comprises one or more of: data regarding meditation practices of the user of the hearing instruments, data regarding physical activity levels of the user of the hearing instruments, or data regarding a respiration rate of the user of the hearing instruments.

### EXAMPLE 1C

A computer-implemented method comprising: obtaining, by a computing system, physiological data based on signals generated by a first set of one or more sensors of a hearing instrument, wherein the physiological data includes heart-related data; modifying, by the computing system, a sensitivity level of a fall detection algorithm based on the physiological data; and performing, by the computing system, the fall detection algorithm to determine, based on signals from a second set of one or more sensors of the hearing instrument, whether a user of the hearing instrument has fallen.

### EXAMPLE 2C

The computer-implemented method of example 1C, wherein the computing system is included in the hearing instrument.

### EXAMPLE 3C

The computer-implemented method of any of examples 1C-2C, wherein the heart-related data comprises one or more of: data indicating a blood pressure of the user of the hearing instrument, data indicating a heart rate of the user of the hearing instrument, electrocardiogram (ECG) data for the user of the hearing instrument, or data regarding a potential cardiac arrythmia of the user of the hearing instrument.

### EXAMPLE 4C

The computer-implemented method of any of examples 1C-3C, wherein the physiological data comprises one or more: electroencephalogram (EEG) data generated by one or more electrodes included in the hearing instrument, data indicating a respiration rate of the user of the hearing instrument, or data indicating a level of physical activity of the user of the hearing instrument.

### EXAMPLE 5C

The computer-implemented method of any of examples 1C-4C, wherein performing the fall detecting algorithm comprises: generating, by the computing system, a confidence value that indicates a level of confidence that the user of the hearing instrument has fallen; and determining, by the computing system, that the user has fallen based on the confidence value being greater than the sensitivity level.

### EXAMPLE 6C

The computer-implemented method of any of examples 1C-5C, further comprising modifying the sensitivity level in response to receiving an indication of user input.

### EXAMPLE 1D

A computer-implemented method comprising: determining, by a computing system, whether a user of a hearing instrument has fallen; based on a determination that the user has fallen, activating, by the computing system, one or more sensors of the hearing instrument that generate heart-related data regarding the user; determining, by the computing system, based on the heart-related data, whether to prompt the user to confirm that the user has fallen; and based on a determination to prompt the user to confirm that the user has fallen, causing, by the computing system, the hearing instrument to generate a message prompting the user to confirm that the user has fallen.

### EXAMPLE 2D

The method of example 1D, wherein: the one or more sensors include a photoplethysmography (PPG) sensor, and determining whether to prompt the user comprises: determining, by the computing system, based on heart-related data generated by the PPG sensor, a heart rate of the user; and determining, by the computing system, based on the heart rate of the user being above a first threshold or below a second threshold, whether to prompt the user the confirm that the user has fallen.

### EXAMPLE 3D

The method of any of examples 1D-2D, wherein: the one or more sensors include a photoplethysmography (PPG) sensor, and determining whether to prompt the user comprises: determining, by the computing system, based on heart-related data generated by the PPG sensor, a level of blood perfusion of the user; and determining, by the computing system, based on the level of blood perfusion of the user, whether to prompt the user the confirm that the user has fallen.

### EXAMPLE 1E

A computing system comprising: a communication unit configured to receive data from one or more hearing instruments; and one or more processors configured to perform the methods of any of examples 1A-3D.

### EXAMPLE 2E

A computing system comprising means for performing the methods of any of examples 1A-3D.

### EXAMPLE 3E

A computer-readable storage medium having instructions stored thereon that, when executed, cause a computing system to perform the methods of any of examples 1A-3D.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A computer-implemented method comprising:
receiving, by a computing system comprising a set of one or more electronic computing devices, heart-related data from one or more hearing instruments;
determining, by the computing system, based on the heart-related data received from the one or more hearing instruments, a heart health measure for a user of the one or more hearing instruments, the heart health measure being an indication of one or more aspects of a health of a heart of the user; and
outputting, by the computing system, an indication of the heart health measure to the user of the hearing instruments.

2. The computer-implemented method of claim 1, wherein:
a particular hearing instrument in the set of hearing instruments is configured to receive a request for the heart-related data and wirelessly transmit the heart-related data in response to the request, wherein the request is initiated by the user of the one or more hearing instruments,
the particular hearing instrument uses electrical energy from a battery internal to the particular hearing instrument to wirelessly transmit the heart-related data to the computing system in response to the request,
determining the heart health measure comprises increasing, by the computing system, a point total of the user by one or more points based on a number of times that the user initiated a request for the heart-related data during a scoring time period; and
the method further comprises:
determining, by the computing system, based on the heart-related data, whether to generate a notification; and
based on a determination to generate the notification, sending, by the computing device, the notification to one or more recipients.

3. The computer-implemented method of claim 2, wherein the one or more recipients include at least one of:
the user of the hearing instruments, or
a third party, wherein the third party is a party other than the user of the hearing instruments and other than a provider of the computing system.

4. The computer-implemented method of claim 1, wherein determining the heart health measure comprises:
determining, by the computing system, a plurality of sub-components of the heart health measure; and
determining, by the computing system, the heart health measure based on the plurality of sub-components of the heart health measure.

5. The computer-implemented method of claim 4, wherein determining the plurality of sub-components comprises one or more of:
determining, by the computing system, a heart rate sub-component, or
determining, by the computing system, a heart rate recovery sub-component.

6. The computer-implemented method of claim 1, further comprising:
determining, by the computing system, based on the data received from the one or more hearing instruments, a body measure for the user, the body measure being an indication of physical health of the user; and
outputting, by the computing system, an indication of the body measure.

7. The computer-implemented method of claim 6, wherein the heart health measure is a sub-component of the body measure.

8. The computer-implemented method of claim 6, further comprising determining a wellness measure based on the body measure and the heart health measure, the wellness measure being an indication of an overall wellness of the user.

9. The computer-implemented method of claim 1, wherein:
the heart-related data from the one or more hearing instruments is based on one or more of:
a signal from a photoplethysmography (PPG) sensor of the one or more hearing instruments,
a signal from an inertial measurement unit (IMU) of the one or more hearing instruments, or
one or more signals from electrocardiogram (ECG) electrodes of the one or more hearing instruments.

10. A computing system comprising:
a communication unit configured to receive data from one or more hearing instruments; and
one or more processors configured to:
receive heart-related data from one or more hearing instruments;
determine, based on the heart-related data received from the one or more hearing instruments, a heart health measure for a user of the one or more hearing instruments, the heart health measure being an indication of one or more aspects of a health of a heart of the user; and
output an indication of the heart health measure to the user of the hearing instruments.

11. The computing system of claim 10, wherein:
a particular hearing instrument in the set of hearing instruments is configured to receive a request for the heart-related data and wirelessly transmit the heart-related data in response to the request, wherein the request is initiated by the user of the one or more hearing instruments,
the particular hearing instrument uses electrical energy from a battery internal to the particular hearing instrument to wirelessly transmit the heart-related data to the computing system in response to the request,
the one or more processors are configured such that, as part of determining the heart health measure, the one or more processors are configured to increase a point total of the user by one or more points based on a number of times that the user initiated a request for the heart-related data during a scoring time period; and
the one or more processors are further configured to:
determine, based on the heart-related data, whether to generate a notification; and
based on a determination to generate the notification, send the notification to one or more recipients.

12. The computing system of claim 11, wherein the one or more recipients include at least one of:
the user of the hearing instruments, or
a third party, wherein the third party is a party other than the user of the hearing instruments and other than a provider of the computing system.

13. The computing system of claim 10, wherein the one or more processors are configured such that, as part of determining the heart health measure, the one or more processors:
determine a plurality of sub-components of the heart health measure; and
determine the heart health measure based on the plurality of sub-components of the heart health measure.

14. The computing system of claim 13, wherein the one or more processors are configured such that, as part of determining the plurality of sub-components, the one or more processors:
determine a heart rate sub-component, or
determine a heart rate recovery sub-component.

15. The computing system of claim 10, wherein the one or more processors are further configured to:
determine, based on the data received from the one or more hearing instruments, a body measure for the user, the body measure being an indication of physical health of the user; and
output an indication of the body measure.

16. The computer system of claim 15, wherein the heart health measure is a sub-component of the body measure.

17. The computing system of claim 15, wherein the one or more processors are further configured to determine a wellness measure based on the body measure and the heart health measure, the wellness measure being an indication of an overall wellness of the user.

18. The computing system of claim 10, wherein:
the heart-related data from the one or more hearing instruments is based on one or more of:
a signal from a photoplethysmography (PPG) sensor of the one or more hearing instruments,
a signal from an inertial measurement unit (IMU) of the one or more hearing instruments, or
one or more signals from electrocardiogram (ECG) electrodes of the one or more hearing instruments.

19. A computer-readable storage medium having instructions stored thereon that, when executed, cause a computing system to:
receive heart-related data from one or more hearing instruments;
determine, based on the heart-related data received from the one or more hearing instruments, a heart health measure for a user of the one or more hearing instruments, the heart health measure being an indication of one or more aspects of a health of a heart of the user; and
output an indication of the heart health measure to the user of the hearing instruments.

20. The computer-readable storage medium of claim 19, wherein:
a particular hearing instrument in the set of hearing instruments is configured to receive a request for the heart-related data and wirelessly transmit the heart-related data in response to the request, wherein the request is initiated by the user of the one or more hearing instruments,
the particular hearing instrument uses electrical energy from a battery internal to the particular hearing instrument to wirelessly transmit the heart-related data to the computing system in response to the request,
as part of causing the computing system to determine the heart health measure, execution of the instructions causes the computing system to increase a point total of the user by one or more points based on a number of times that the user initiated a request for the heart-related data during a scoring time period; and
execution of the instructions further causes the computing system to:
determine, based on the heart-related data, whether to generate a notification; and
based on a determination to generate the notification, send the notification to one or more recipients.
